(19) United States
(12) Patent Application Publication
(10) Pub. No.: US 2023/0071042 A1
(43) Pub. Date: Mar. 9, 2023

(54) BCR TRANSGENIC MICE WITH A COMMON LEADER SEQUENCE

(71) Applicant: BRISTO-MYERS SQUIBB COMPANY, Princeton, NJ (US)

(72) Inventors: Ralston M. BARNES, San Francisco, CA (US); Arvind RAJPAL, San Francisco, CA (US); Ailan LU, Palo Alto, CA (US); Gabriel WU, Newark, CA (US); John M. MAJERCAK, Wayne, PA (US)

(21) Appl. No.: 17/785,180

(22) PCT Filed: Dec. 17, 2020
(86) PCT No.: PCT/US2020/065450

§ 371 Date: Jun. 14, 2022

Related U.S. Application Data
(60) Provisional application No. 62/949,707, filed on Dec. 18, 2019.

(51) Int. Cl. A01K 67/027; C07K 16/00

(52) U.S. Cl. CPC A01K 67/0278; C07K 16/00; A01K 2227/105; A01K 2267/01; C07K 2317/21; C07K 2317/56

(57) ABSTRACT

The present invention provides transgenic animals comprising some or all components of a human heavy and/or light chain immunoglobulin variable region locus, methods of making such animals, methods of making human antibodies using such animals, and methods of treatment using the human antibodies made in such animals, wherein the animals comprise in their genome a plurality of human heavy chain V gene segments all of which are immediately preceded by the same first leader peptide-encoding sequence, and/or a plurality of human light chain V gene segments all of which are immediately preceded by the same second leader peptide-encoding sequence, or both. The invention also provides polynucleotide constructs comprising two or more human heavy or light chain leader/V gene segments comprising identical leader peptide-encoding sequences. Such animals, constructs and methods find use in efficient generation of optimally diverse populations of antibodies against antigens of interest, such as antigens of therapeutic interest.

### CROSS REFERENCE TO RELATED APPLICATIONS

This application claims priority to U.S. Provisional Application No. 62/949,707, filed 18 Dec. 2019, the disclosure of which is incorporated herein by reference.

### SEQUENCE LISTING

The Sequence Listing filed electronically herewith is also hereby incorporated by reference in its entirety (File Name: 20201120_SEQL_13330WOPCT_GB.txt; Date Created: 20 Nov. 2020; File Size: 29 KB).

### BACKGROUND OF THE INVENTION

Antibodies are increasingly used as medicines to treat human diseases, such as autoimmune disease and cancer. The earliest such therapeutic antibodies were non-human (e.g. mouse) antibodies that elicited a human anti-drug antibody response (e.g. human anti-mouse antibodies—HAMA response), and thus could not be dosed repeatedly. Subsequent generations of therapeutic antibodies were “chimeric” forms of non-human, typically mouse, antibodies in which human constant regions are substituted for the non-human constant regions, and “humanized” antibodies in which all sequences other than the complementarity determining regions (CDRs) are converted to human counterparts to minimize immunogenicity in human subjects.

The most recent generation of therapeutic antibodies comprise sequences derived completely from human germline immunoglobulin sequences, for example in vivo from transgenic mice humanized at immunoglobulin loci, or in vitro from phage display libraries. Finlay & Almagro (2012) Front. Immunol. 3(342):1. Transgenic mice harboring human immunoglobulin gene segments are immunized with antigens designed to elicit antibodies to targets of therapeutic interest. A diverse selection of antibody sequences in the resulting pool of polyclonal anti-antigen antibodies, including antibodies derived from the greatest number of different germline sequences, maximizes the chance of finding antibodies with superior properties, e.g. high target affinity and epitope diversity. Antibody sequence diversity is dependent on the number of different sequence elements, such as V, D and J gene segments for heavy chains, a V and J gene segments for light chains, available in the mouse germline for incorporation into heavy and light chains, as well as addition/deletion of nucleotides in CDR3 during rearrangement, and somatic hypermutation occurring during affinity maturation of the antibodies during B cell development. Different V, D and J gene segments, however, are incorporated at different frequencies into antibodies leading to skewing of antibody sequences toward preferred germline sequences, limiting sequence diversity. Finlay & Almagro (2012) Front. Immunol. 3:242. Different leader peptides associated with different V gene segments may also affect translation and secretion of particular antibodies derived from those sequences, further skewing the distribution of antibody sequences and limiting diversity.

Antibodies generated by the mice must then be recovered for selection of a preferred antibody, such as a therapeutic candidate. Regardless of the diversity of antibodies generated by the mouse, only those that are efficiently recovered will be available, e.g., for selection as therapeutic candidates. Any isolation step that biases toward some antibodies at the expense of others will further lower the diversity of the pool of polyclonal antibodies.

The need exists for improved methods for obtaining pools of polyclonal antibodies that enhance the diversity of antibodies from which the most desirable may be selected, and for improved mice for use in such methods. Ideally, such methods would avoid bias in generation, isolation and evaluation of antibodies from an initial pool of polyclonal antibodies so as not to lose good candidates before they can even be tested.

### SUMMARY OF THE INVENTION

The present invention provides a non-human animal with a humanized heavy chain immunoglobulin locus for use in producing human antibodies, wherein the humanized heavy chain immunoglobulin locus comprises a plurality of human heavy chain leader/V gene segments all comprising the same leader peptide-encoding sequence. The invention also provides a non-human animal with a humanized light chain immunoglobulin locus for use in producing human antibodies, wherein the humanized immunoglobulin light chain locus comprises a plurality of human light chain leader/V gene segments all comprising the same leader peptide-encoding sequence. The invention further provides a non-human animal with humanized heavy and light chain immunoglobulin loci, wherein the heavy chain locus comprises a plurality of human heavy chain leader/V gene segments all comprising the same first leader peptide-encoding sequence, and the light chain locus comprises a plurality of human light chain leader/V gene segments all comprising the same second leader peptide-encoding sequence. In some embodiments, the first leader peptide-encoding sequence is different from the second leader peptide-encoding sequence, and in other embodiments they are the same sequence. In various embodiments, the animal is a mouse, a rat, or a cow.

In another aspect, the invention provides a method of making the transgenic non-human animals of the preceding paragraph, comprising integrating into the genome of a non-human animal a plurality of human heavy chain leader/V gene segments all comprising the same first leader peptide-encoding sequence and/or a plurality of human light chain leader/V gene segments all comprising the same second leader peptide-encoding sequence, wherein the first and second leader peptide-encoding sequences are optionally the same sequence. In some embodiments, no additional human heavy or light chain leader/V gene segments are introduced, or are present in, the genome of the non-human animal, other than the plurality of human heavy or light chain leader/V gene segments comprising the same first leader peptide-encoding sequence. In some embodiments, the transgenic non-human animal comprises both the plurality of human heavy chain leader/V gene segments all comprising the same first leader peptide-encoding sequence and the plurality of human light chain leader/V gene segments all comprising the same second leader peptide-encoding sequence, wherein the first and second leader peptide-encoding sequences are optionally the same sequence. In various embodiments, the animal is a mouse, a rat, or a cow.

In another aspect, the invention provides methods of making a human antibody or antigen-binding fragment thereof, to an antigen of interest comprising immunizing mice having humanized heavy and light chain immunoglobulin loci, wherein all heavy chain leader/V gene segments comprise the same first leader peptide-encoding sequence and all light chain leader/V gene segments comprise the same second leader peptide-encoding sequence, with an antigen, or an antigenic fragment or derivative thereof, and recovering from said mice a human antibody that specifically binds to the antigen, or the sequence encoding the heavy and light chain variable regions of the human antibody that specifically binds to the antigen. The recovery may be by the hybridoma method, single B-cell cloning, or any other suitable method of obtaining the antibody or its encoding nucleic sequence, from cells of a non-human animal expressing the antibody. In some embodiments, the leader peptide-encoding sequences for the heavy and light chain leader/V gene segments are selected from the sequences listed in Tables 2, 3 and 4.

In yet a further aspect, the invention provides human antibodies made by the process of the preceding paragraph.

In yet a further aspect, the invention provides methods of treatment of subjects, e.g. human subjects, comprising administration of human antibodies made by the methods of the preceding paragraph. In various embodiments, the invention provides treatment of subject having an autoimmune disease, an infectious disease, a cardiovascular disease, or cancer.

In various embodiments of the animals and methods of the invention, the leader peptide-encoding sequence preceding the heavy chain V gene segments encodes a leader peptide sequence selected from the group consisting of SEQ ID NOs: 71-133 and 135. In another embodiment, the leader peptide-encoding sequence preceding the light chain V gene segments encodes a leader peptide sequence selected from the group consisting of SEQ ID NOs: 71-133 and 135. In yet another embodiment, the leader peptide-encoding sequences preceding both the heavy and light chain V gene segments are selected from the sequences indicated in the preceding two sentences, respectively. In specific embodiments, the heavy chain leader peptide-encoding sequence encodes a leader peptide sequence selected from the group consisting of SEQ ID NOs: 71, 85, 86 and 93 and/or the light chain leader peptide-encoding sequence encodes a leader peptide sequence selected from the group consisting of SEQ ID NOs: 104 and 112, such as the heavy chain leader peptide sequence of SEQ ID NO: 86 (IGHV 3-23) and the light chain leader peptide sequence of SEQ ID NO: 112 (IGKV 3-20).

In various embodiments of the animals and methods of the invention, the leader peptide-encoding sequence preceding the heavy chain V gene segments is a sequence selected from the group consisting of SEQ ID NOs: 1-70, 134, 136 and 137. In another embodiment, the leader peptide-encoding sequence preceding the light chain V gene segments is a sequence selected from the group consisting of SEQ ID NOs: 1-70, 134, 136 and 137. In yet another embodiment, the leader peptide-encoding sequences preceding both the heavy and light chain V gene segments are selected from the sequences indicated in the preceding two sentences, respectively. In specific embodiments, the heavy chain leader peptide-encoding sequence is selected from the group consisting of SEQ ID NOs: 1, 15, 16, 27 and 136 and/or the light chain leader peptide-encoding sequence is selected from the group consisting of SEQ ID NOs: 39, 49, and 137, such as the heavy chain leader peptide-encoding sequence of SEQ ID NO: 16 or 136 (IGHV 3-23 or IGHV 3-23 genomic) and the light chain leader peptide-encoding sequence of SEQ ID NO: 49 or 137 (IGKV 3-20 or IGKV 3-20 genomic). In a preferred embodiment the genomic leader peptide-encoding sequences of IGHV 3-23 (SEQ ID NO: 136) and IGKV 3-20 (SEQ ID NO: 137) are used for heavy and light chain V gene segments, respectively.

In another aspect, the invention provides polynucleotides comprising a plurality (two or more) of heavy or light chain V gene segments preceded by a common (identical) leader peptide-encoding sequence. In some embodiments the polynucleotides comprise a plurality of naturally occurring human heavy chain V gene segments with a single leader peptide-encoding sequence encoding a leader peptide sequence selected from the group consisting of SEQ ID NOs: 71-133 and 135, such as SEQ ID NOs: 71, 85, 86 or 93, immediately upstream. In other embodiments the polynucleotides comprise a plurality of naturally occurring human light chain V gene segments with a single leader peptide-encoding sequence encoding a leader peptide sequence selected from the group consisting of SEQ ID NOs: 71-133 and 135, such as SEQ ID NOs: 104 or 112, immediately upstream. Such polynucleotides may be, for example, synthetic, or integrated into longer polynucleotide constructs, such as vectors, or integrated into chromosomes.

Exemplary heavy chain leader peptide-encoding sequences for inclusion in the polynucleotides of the present invention include SEQ ID NOs: 1, 15, 16, 27 and 136, such as SEQ ID NOs: 16 and 136, and more specifically SEQ ID NO: 136. Exemplary light chain leader peptide-encoding sequences for inclusion in the polynucleotides of the present invention include SEQ ID NOs: 39, 49, and 137, such SEQ ID NOs: 49 and 137, and more specifically SEQ ID NO: 137.

In various embodiments, the transgenic animals or polynucleotides of the present invention comprise, or the methods of present invention involve use of, a plurality of heavy and/or light chain V gene segments selected from the group consisting of all naturally occurring human V gene segments, or a subset thereof, such as human heavy chain V gene segments IGHV 3-23; IGHV 5-51; IGHV3-7; IGHV 1-2; IGHV 1-69-1; IGHV 3-48; IGHV 1-18; IGHV 1-46; IGHV 3-21; IGHV 3-30; IGHV 3-74; IGHV 4-39; IGHV 3-9; IGHV 2-5; IGHV 1-3; IGHV 4-4; IGHV 7-4-1; IGHV 3-66; and IGHV 1-24 and/or human light chain V gene segments IGKV 1-39; IGKV 3-11; IGKV 1-33; IGKV 3-20; IGKV 4-1; IGKV 1-27; IGKV 1-5; IGKV 1-16; IGKV 1-12; IGKV 2-30; IGKV 3-15; IGKV 2-28; IGKV 1D-13; IGKV 1-17; IGKV 6-21; IGKV 1-9; and IGKV 1D-43. In other embodiments the plurality of heavy and/or light chain human V gene segments includes one or more non-naturally occurring V gene segments, such as an engineered or mutant V gene segment. In various embodiments, one or more human J gene segments, and one or more human D gene segments in the case of heavy chains, are included in the transgenic animals or polynucleotides, such as all naturally occurring D and/or J gene segments, or a desired subset thereof. In other embodiments non-naturally occurring D and/or J gene segments are included, such as engineered or mutant D and/or J gene segments.

In a different aspect, the invention provides non-human animals, and methods of making those animals, with a humanized heavy chain immunoglobulin locus wherein the humanized heavy chain immunoglobulin locus comprises a plurality of human heavy chain leader/V gene segments comprising more than one, but a limited number of, leader peptide-encoding sequences. Such animals may comprise two, three or more different leader peptide-encoding sequences associated with the various V gene segments, but at least two of the leader/V gene segments comprise the same leader peptide-encoding sequence. Such embodiments do not achieve the maximal benefits of using a single leader peptide-encoding sequence for all leader/V gene segments, and may require use of a mixture of primers for amplification, but may still exhibit substantial advantages over use of the different naturally associated leader peptide-encoding with each V gene segment. Use of more than one leader peptide encoding sequence may be necessary, for example, if no single leader peptide-encoding sequence works well with all desired V gene segments. Although the bulk of the specification relates to embodiments comprising a single human genomic variable region immunoglobulin sequences for all heavy and/or light chain V gene segments, one of skill in the art would recognize that the benefits of the invention may be achieved in large part even if more than one leader peptide-encoding sequence is used, with the lowest number of sequences being preferred.

### BRIEF DESCRIPTION OF THE DRAWINGS

FIG. 1 provides a schematic representation of a human immunoglobulin heavy chain variable domain locus as found in current transgenic animals used to generate human antibodies. FIG. 1 does not represent any aspect of the present invention, and is provided only to illustrate some of the deficiencies of current methods. An array of gene segments for a heavy chain variable domain locus is shown, with individual leader/V, D and J gene segments represented by rectangular boxes. All leader/V gene segment boxes represent both the V gene sequence and the naturally associated leader peptide-encoding sequence immediately upstream thereof. Leader/V gene segments are shown as boxes with different fill patterns to represent distinct leader-peptide encoding sequences for each leader/V gene segment, rather than the different V gene sequence. The number of rectangular boxes does not represent any specific number of gene segments for any of the leader/V, D, and J gene segments. FIG. 1 further provides schematic representations of two PCR reactions (PCR #1 and PCR #2). For the purposes of the illustrations in the figures only, “Fc” refers to the entire constant region sequence (CH1, hinge, CH2 and CH3) and not just CH2 and CH3 domains. “FR1” refers to framework 1 at the amino-terminus of the mature heavy chain variable region, which is immediately downstream of the leader peptide-encoding sequence. Primers are named for the PCR reaction in which they are used and whether they are forward (“for”) or reverse (“rev”) primers. Mixtures of primers used for primer 1-for and primer 2-for are illustrated as a mixture of arrows with different line patterns. “Optimal promoter and leader” and “desired Fc” refer to additional genetic elements in the vector into which the amplified variable region sequence is cloned.

FIG. 2 provides a schematic representation of a new and improved human immunoglobulin heavy chain variable domain locus a for use in transgenic animals used to generate human antibodies. All leader/V gene segment boxes represent both the V gene sequence and the common leader peptide-encoding sequence immediately upstream thereof. All leader/V gene segments are the same color (black) to indicate that they comprise the same leader peptide-encoding sequence, even though each leader/V gene segment comprises a different V gene sequence. Unlike FIG. 1, primer 1-for and primer 2-for are single primers, not mixtures of primers, and primer 2-for is complementary to sequence in the leader peptide-encoding region rather than variable region (FR1). Nomenclature is otherwise as in FIG. 1. The details of FIG. 2 do not limit the present invention, and FIG. 2 is provided merely to illustrate the principle of the invention schematically, and specifically one embodiment thereof.

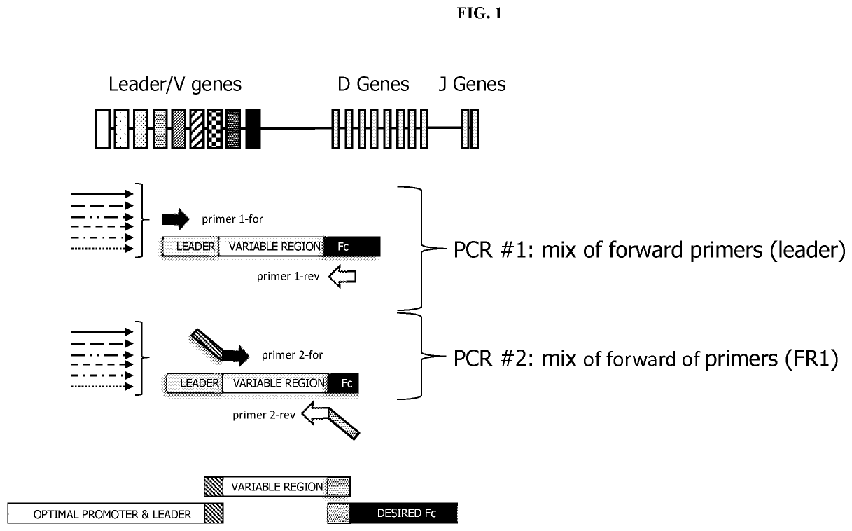

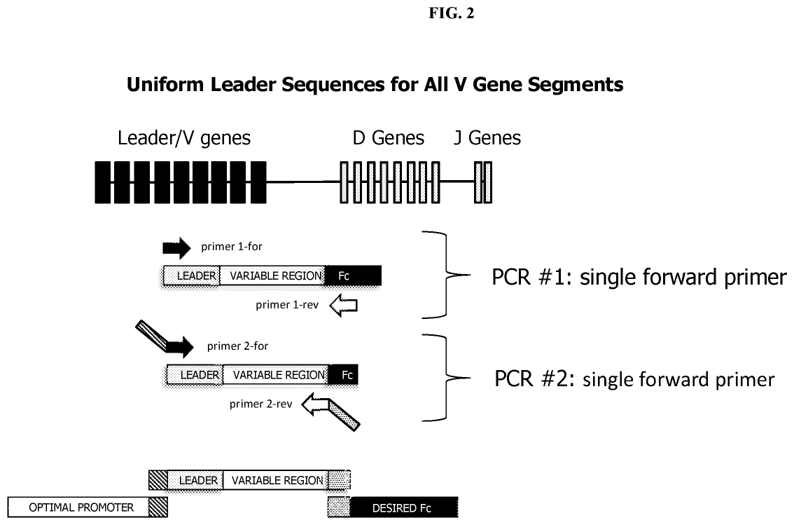

Schematics analogous to FIGS. 1 and 2 but for light chain constructs, i.e. simply omitting D gene segments, would be immediately apparent to those of skill in the art.

### DETAILED DESCRIPTION OF THE INVENTION

### Definitions

In order that the present disclosure may be more readily understood, certain terms are first defined. As used in this application, except as otherwise expressly provided herein, each of the following terms shall have the meaning set forth below. Additional definitions are set forth throughout the application.

“Administering” refers to the physical introduction of a composition comprising an agent, such as an antigen or a therapeutic agent, to a subject, using any of the various methods and delivery systems known to those skilled in the art. Preferred routes of administration for antibodies of the invention include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody of the invention can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

Administration may be performed by one or more individual, including but not limited to, a doctor, a nurse, another healthcare provider, or the patient himself or herself.

“Animal,” as used herein with reference to transgenic animals comprising optimized human immunoglobulin loci for use in the methods of the present invention, refers to any animal species suitable for production of human antibodies. Exemplary animals that have been used to produce human antibodies include rodents, such as mice and rats, and cows. See, e.g., Bruggemann et al. (2015) Arch Immunol Ther Exp (Warsz) 63:101. Other animals may also be used. Unless otherwise indicated, methods and examples provided herein with specific reference to mice would be equally applicable to other suitable animal species.

An animal is “transgenic,” for the purposes of the present disclosure, if its germline nucleic acid sequences are modified to comprise nucleic acid sequences derived from a different species, such as sequences derived from human germline sequences, or artificial sequences not found in the mouse genome. Transgenic animals of the present invention, such as transgenic mice, will typically comprise human immunoglobulin sequences integrated into their genomes. The heterologous nucleic acid sequences may be introduced at any locus, e.g. at the corresponding animal immunoglobulin locus, and may be introduced by any method.

“Introduction” of a genetic construct into an animal, such as a mouse, may comprise breeding or crossing of animals having desired traits to create offspring harboring both traits. For example, animals harboring a transgenic human heavy chain variable region locus may be crossed with animals harboring a transgenic human light chain variable region locus to create an animal capable of producing antibodies comprising a human variable domain.

An “antibody” (Ab) shall include, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three domains, C_{H1}, C_{H2} and C_{H3}. Each light chain comprises a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. Variable regions may be referred to equivalently herein as “variable domains,” and constant regions may be referred to equivalently herein as “constant domains.”

“Antibodies,” depending on the context, may refer to either a plurality or a limited number of individual antibodies, or a pool of polyclonal antibodies, such as the pool of all anti-antigen antibodies recovered from an immunization with an antigen.

As used herein, and in accord with conventional interpretation, an antibody that is described as comprising “a” heavy chain and/or “a” light chain refers to antibodies that comprise “at least one” of the recited heavy and/or light chains, and thus will encompass antibodies having two or more heavy and/or light chains. Specifically, antibodies so described will encompass conventional antibodies having two substantially identical heavy chains and two substantially identical light chains. Antibody chains may be substantially identical but not entirely identical if they differ due to post-translational modifications, such as C-terminal cleavage of lysine residues, alternative glycosylation patterns, etc.

Unless indicated otherwise or clear from the context, an antibody defined by its target specificity (e.g. an “anti-CTLA-4 antibody”) refers to antibodies that can bind to its human target (e.g. human CTLA-4). Such antibodies may or may not bind to CTLA-4 from other species.

The immunoglobulin may derive from any of the commonly known isotypes, including but not limited to IgA, secretory IgA, IgG and IgM. The IgG isotype may be divided in subclasses in certain species: IgG1, IgG2, IgG3 and IgG4 in humans, and IgG1, IgG2a, IgG2b and IgG3 in mice. IgG antibodies may be referred to herein by the symbol gamma (γ) or simply “G,” e.g. IgG1 may be expressed as “γ1” or as “G1,” as will be clear from the context. “Isotype” refers to the antibody class (e.g., IgM or IgG1) that is encoded by the heavy chain constant region genes. “Antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies.

An “isolated antibody” refers to an antibody that is substantially free of other antibodies having different antigenic specificities (e.g., an isolated antibody that binds specifically to CTLA-4 is substantially free of antibodies that bind specifically to antigens other than CTLA-4). An isolated antibody that binds specifically to CTLA-4 may, however, cross-react with other antigens, such as CTLA-4 molecules from different species. Moreover, an isolated antibody may be substantially free of other cellular material and/or chemicals. By comparison, an “isolated” nucleic acid refers to a nucleic acid composition of matter that is markedly different, i.e., has a distinctive chemical identity, nature and utility, from nucleic acids as they exist in nature. For example, an isolated DNA, unlike native DNA, is a free-standing portion of a native DNA and not an integral part of a larger structural complex, the chromosome, found in nature. Further, an isolated DNA, unlike native DNA, can be used as a PCR primer or a hybridization probe for, among other things, measuring gene expression and detecting biomarker genes or mutations for diagnosing disease or predicting the efficacy of a therapeutic. An isolated nucleic acid may also be purified so as to be substantially free of other cellular components or other contaminants, e.g., other cellular nucleic acids or proteins, using standard techniques well known in the art.

The term “monoclonal antibody” (“mAb”) refers to a preparation of antibody molecules of single molecular composition, i.e., antibody molecules whose primary sequences are essentially identical, and which exhibits a single binding specificity and affinity for a particular epitope. Monoclonal antibodies may be produced by hybridoma, recombinant, transgenic or other techniques known to those skilled in the art.

As used herein, a “human” antibody (or antigen binding fragment thereof) refers to an antibody (or fragment) derived from human genomic variable region immunoglobulin sequences, including naturally occurring germline sequences and variants thereof such as variants comprising V gene segments preceded by identical leader peptide-encoding sequences according to the present invention. Derivatives of human genomic variable region immunoglobulin sequences include, e.g., minor sequence changes to eliminate potential amino acid sequence liabilities in the resulting antibodies. A “human” antibody is to be distinguished from an antibody derived from animal, e.g. mouse, germline immunoglobulin sequences. An antibody created in a mouse of the present invention, or generated from a mouse of the present invention, will comprise only human-derived immunoglobulin variable region sequences regardless of the origin of the leader peptide sequence used (human, non-human, artificial) because the leader peptide is cleaved from the mature heavy and light chains of the antibody. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. In the context of a potential human therapeutic antibody isolated from a transgenic animal, an antibody may be considered to be a human antibody if the variable domain is of human origin, regardless of the origin of the constant domain, since the constant domain will be changed to a human constant domain in the final therapeutic antibody anyway. A “fully human” antibody comprises both variable domain and constant domain sequences derived from human germline immunoglobulin sequences.

An “antibody fragment” refers to a portion of a whole antibody, generally including the “antigen-binding portion” (“antigen-binding fragment”) of an intact antibody which retains the ability to bind specifically to the antigen bound by the intact antibody, or the Fc region of an antibody which retains FcR binding capability. Exemplary antibody fragments include Fab fragments and single chain variable domain (scFv) fragments.

“Leader peptide sequence,” as used herein, refers to a stretch of amino acid residues at the N-terminus of a newly synthesized polypeptide that is typically cleaved prior to, or concurrent with, secretion of the protein. A leader peptide sequence may also be referred to, e.g., as a leader sequence, a leader peptide, a signal sequence, and a signal peptide. A leader peptide sequence is typically 16 to 30 amino acids long. For proteins having a leader peptide, the full-length form of the sequence including the leader peptide sequence is referred to as the pro-protein, whereas the sequence remaining after leader peptide removal is referred to as the mature protein. At the genetic level, the leader peptide-encoding sequence is present immediately upstream of sequence encoding framework region 1 (FR1) of the variable domain, found at the 5′ end of the V gene segment.

A “leader peptide-encoding sequence” is a nucleic acid sequence, typically a DNA sequence when referencing genetic constructs for incorporation into, or incorporated into, a non-human animal's genome that encodes a leader peptide sequence. A leader peptide-encoding sequence may be, e.g., the naturally occurring DNA sequence encoding the leader peptide sequence in its organism of origin, with or without any intron sequence that may be naturally present in the leader peptide-encoding sequence in the genome, a codon-optimized DNA sequence encoding the leader peptide sequence, or any other DNA sequence encoding the leader peptide sequence.

“V gene segment,” as used herein, refers to a genomic genetic element that when placed in an immunoglobulin locus, is capable of being rearranged to comprise a sequence encoding the amino-terminal portion of the variable domain of a mature antibody heavy or light chain. Unless otherwise indicated, “V gene segment” refers to a naturally occurring human genomic gene segment, not including the sequence encoding the leader peptide. Janeway et al. (2001) Immunobiology, 5^{th} Ed. at Section 4.2 and FIG. 4.2. “D gene region” and “J gene region” refer to additional genomic genetic elements in the immunoglobulin locus (only “J gene region” in light chains) that are capable of being rearranged to comprise a sequence encoding the carboxy-terminal portion of the variable domain of an antibody heavy or light chain. A transgenic animal of the present invention comprises in its genome one or more human immunoglobulin loci comprising a plurality of V gene segments (two or more), as well as at least one D gene segment (for heavy chains) and at least one J gene segment. “Variable region gene segments” refers collectively to V, D (for heavy chains) and J gene segments as found in a variable region locus.

“Leader/V gene segment,” as used herein, refers to a genetic element comprising a V gene segment immediately downstream of a sequence encoding a leader peptide. When incorporated into a rearranged immunoglobulin locus, the leader/V gene segment is transcribed into RNA and optionally spliced to create the 5′ end of an mRNA encoding the heavy or light chain of an antibody. The leader/V gene segment encodes the leader peptide and the N-terminal portion of a variable domain of the antibody chain, including framework 1 (FR1), complementarity determining region 1 (CDR1), FR2, CDR2, FR3 and the start of CDR3. The leader peptide is cleaved from the heavy and light chains in the mature secreted antibody. In some embodiments of the present invention the V gene sequences in these leader/V gene segments comprise human germline V gene sequences, whereas the leader peptide-encoding sequences may be of human, non-human, or synthetic origin. See, e.g., Tables 2, 3 and 4. In embodiments of the present invention, for a given heavy or light chain locus, the different leader/V gene segments each comprise a different V gene sequence but the same leader peptide-encoding sequence. Unless otherwise indicated, transcribed nucleic acid sequences immediately upstream of a given leader/V gene segment comprise the 5′ UTR (untranslated region) naturally associated with that specific V gene segment in the species of origin, such as human.

An “immunoglobulin locus,” such as a “human immunoglobulin locus,” as used herein, refers to a genomic location comprising nucleic acid sequences necessary to support rearrangement to produce antibody heavy or light chains. In the context of the methods of the invention, or the transgenic animals of the invention, a human heavy or light chain immunoglobulin locus may be located in the animal genome at or near the corresponding human heavy or light chain animal genetic locus, respectively, e.g. near a mouse immunoglobulin locus. A human immunoglobulin locus in a transgenic animal comprises human genetic elements for the variable region of the heavy or light chain (a “human immunoglobulin variable region locus”), and may optionally include one or more human genetic elements for the constant region of the antibody chains, e.g. V, D and J elements for the heavy chain variable region and V and J elements for the light chain variable region. An immunoglobulin locus may comprise any number of V, D or J gene segments, from one up to the naturally occurring number of functional human gene segments, or more. Embodiments of the present invention comprise at least two V gene segments for at least the heavy or light chain locus.

“Sequence,” as used herein, unless otherwise indicated or clear from the context, refers to a nucleic acid sequence, such as a DNA sequence, such as a genomic DNA sequence. “Identical sequences” will therefor necessarily encode the same polypeptide sequences. Unless otherwise indicated, or clear from the context, all references to nucleic acid sequences (such as genes or gene segments) or proteins herein relate to the human (Homo sapiens) orthologs of that nucleic acid sequence or protein.

“Identical leader peptide-encoding sequence,” as used herein with reference to heavy or light chain leader/V gene segments, refers to N-terminal leader peptide-encoding sequences in a plurality of leader/V gene segments that comprise the same nucleic acid sequence as the leader peptide-encoding sequence in all other leader/V gene segments in the heavy chain, or the light chain, respectively. The leader peptide-encoding sequences may be the natural coding sequence for the leader peptide, such as the natural coding sequence for leader peptides derived from specific human V gene segments, or they may be optimized (such as codon optimized) or otherwise altered coding sequences encoding the same leader peptide amino acid sequence, provided they are all the same nucleic acid sequence. In the animals and methods of the present invention, all heavy chain leader/V gene segments will comprise identical leader peptide-encoding sequences (referred to as a “first” leader peptide-encoding sequence), and all light chain leader/V gene segments will comprise identical leader peptide-encoding sequences (referred to as a “second” leader peptide-encoding sequence). The first leader peptide-encoding sequence may or may not be the same as the second leader peptide-encoding sequence.

A sequence element is “upstream” of a second sequence element if it is closer to the 5′ end of a coding strand of a polynucleotide, or closer to the amino terminus (N-terminus) of a polypeptide, than the second sequence. A sequence element is “immediately upstream of” a second sequence element if directly fused to the 5′ end (or N-terminus) of the second sequence element with no additions or deletions at the junction. Analogously, a sequence element is “downstream” of a second sequence element if it is closer to the 3′ end of a coding strand of a polynucleotide, or closer to the carboxy terminus (C-terminus) of a polypeptide, than the second sequence element, and “immediately downstream” if it is fused directly to the 3′ end (or C-terminus) of the second sequence element with no additions or deletions.

“Genomic” sequences, as used herein, are sequences found in chromosomes of germline cells of an animal, such as a human (Homo sapiens) or a non-human transgenic animal of the present invention. Genomic sequences may or may not comprise introns. “Intronless” sequences, as used herein, are sequences in which there are no introns, and thus encode a protein directly without need for splicing, and thus correspond to the spliced mRNA sequence for that protein or equivalently the cDNA sequence for that protein. A sequence is typically referred to as intronless if it is derived from a genomic sequence that comprises one or more introns. In various embodiments, some or all leader peptide-encoding sequences and V gene segments used in the transgenic animals, methods and nucleic acids of the present invention are intronless sequences. In other embodiments introns are retained in the leader peptide-encoding sequences and/or the V gene segments.

Unless otherwise indicated, discussions of sequences or gene segments herein refer to their copy number in the haploid genome, i.e. on an individual chromosome in one genome complement. Similarly, discussions of sequences or gene segments herein do not take into consideration heterozygosity. Unless otherwise indicated, animals of the present invention may be either heterozygous or homozygous for a given sequence or gene element. An animal that is heterozygous for a given sequence or gene element may be selectively bred to generate progeny animals that are homozygous, for example for use in raising antibodies to an antigen of interest.

“Cancer” refers a broad group of various diseases characterized by the uncontrolled growth of abnormal cells in the body. Unregulated cell division and growth divide and grow results in the formation of malignant tumors or cells that invade neighboring tissues and may also metastasize to distant parts of the body through the lymphatic system or bloodstream.

A “cell surface receptor” refers to molecules and complexes of molecules capable of receiving a signal and transmitting such a signal across the plasma membrane of a cell.

An “effector cell” refers to a cell of the immune system that expresses one or more FcRs and mediates one or more effector functions. Preferably, the cell expresses at least one type of an activating Fc receptor, such as, for example, human FcγRIII, and performs ADCC effector function. Examples of human leukocytes which mediate ADCC include peripheral blood mononuclear cells (PBMCs), NK cells, monocytes, macrophages, neutrophils and eosinophils.

“Effector function” refers to the interaction of an antibody Fc region with an Fc receptor or ligand, or a biochemical event that results therefrom. Exemplary “effector functions” include Clq binding, complement dependent cytotoxicity (CDC), Fc receptor binding, FcγR-mediated effector functions such as ADCC and antibody dependent cell-mediated phagocytosis (ADCP), and down-regulation of a cell surface receptor (e.g., the B cell receptor; BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) receptors and one inhibitory (FcγRIIB) receptor. Various properties of human FcγRs are summarized in Table 1. The majority of innate effector cell types co-express one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans.

An “Fc region” (fragment crystallizable region) or “Fc domain” or “Fc,” except as used in the figures, refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, the Fc region is a polypeptide comprising the constant region of an antibody excluding the first constant region immunoglobulin domain. In IgG, IgA and IgD antibody isotypes, the Fc region is composed of two identical protein fragments, derived from the second (C_{H2}) and third (C_{H3}) constant domains of the antibody's two heavy chains; IgM and IgE Fc regions contain three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc or a variant Fc. Fc may also refer to this region in isolation or in the context of an Fc-comprising protein polypeptide such as a “binding protein comprising an Fc region,” also referred to as an “Fc fusion protein” (e.g., an antibody or immunoadhesion).

TABLE 1
Properties of Human FcγRs

| | Allelic | Affinity for | Isotype | |
|---|---|---|---|---|
| Fcγ | variants | human IgG | preference | Cellular distribution |
| FcγRI | None | High (K_{D} | IgG1 = 3 > | Monocytes, |
| | described | ~10 nM) | 4 >> 2 | macrophages, |
| | | | | activated neutrophils, |
| | | | | dendritic cells? |
| FcγRIIA | H131 | Low to | IgG1 > 3 > | Neutrophils, |
| | | medium | 2 > 4 | monocytes, |
| | R131 | Low | IgG1 > 3 > | macrophages, |
| | | | 4 > 2 | eosinophils, |
| | | | | dendritic cells, |
| | | | | platelets |
| FcγRIIIA | V158 | Medium | IgG1 = 3 >> | NK cells, monocytes, |
| | | | 4 > 2 | macrophages, |
| | F158 | Low | IgG1 = 3 >> | mast cells, |
| | | | 4 > 2 | eosinophils, |
| | | | | dendritic cells? |
| FcγRIIB | I232 | Low | IgG1 = 3 = | B cells, monocytes, |
| | | | 4 > 2 | macrophages, |
| | T232 | Low | IgG1 = 3 = | dendritic cells, |
| | | | 4 > 2 | mast cells |

An “immune response” refers to a biological response within a vertebrate against foreign agents, which response protects the organism against these agents and diseases caused by them. The immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues.

An “immunomodulator” or “immunoregulator” refers to a component of a signaling pathway that may be involved in modulating, regulating, or modifying an immune response. “Modulating,” “regulating,” or “modifying” an immune response refers to any alteration in a cell of the immune system or in the activity of such cell. Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified, some of which may have enhanced function in a tumor microenvironment. In preferred embodiments of the disclosed invention, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

A “protein” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. The term “protein” is used interchangeable herein with “polypeptide.”

Unless otherwise indicated, or clear from the context, a “subject” refers to a human being to which a therapeutic substance is administered. The term “non-human animal” includes, but is not limited to, vertebrates such as nonhuman primates, sheep, dogs, rabbits, rodents such as mice, rats and guinea pigs, avian species such as chickens, amphibians, and reptiles. In preferred embodiments, the subject is a mammal such as a nonhuman primate, sheep, dog, cat, rabbit, ferret or rodent. In more preferred embodiments of any aspect of the disclosed invention, the subject is a human. The terms, “subject” and “patient” are used interchangeably herein.

“Treatment” or “therapy” of a subject refers to any type of intervention or process performed on, or administering an active agent to, the subject with the objective of reversing, alleviating, ameliorating, inhibiting, slowing down or prevent the onset, progression, development, severity or recurrence of a symptom, complication, condition or biochemical indicia associated with a disease.

### Common Leader Peptide Approach

The present invention provides methods of making transgenic animals for use in generating diverse polyclonal pools of human antibodies, e.g. for selection of antibodies for use as human therapeutics. The invention also provides transgenic animals, such as mice, comprising improved artificial human immunoglobulin loci for use in generating diverse polyclonal pools of human antibodies, methods of making antibodies using such transgenic animals, antibodies obtained from such transgenic animals, methods of treatment using antibodies obtained from such transgenic animals, and related polynucleotide constructs. The methods of the invention involve introduction of a plurality of genomic heavy and/or light chain leader/V gene segments into an animal, e.g. a mouse, rat or cow, in which all leader/V gene segments for a given chain comprise identical leader peptide-encoding sequences.

The human immunoglobulin heavy chain locus comprises approximately 45 functional V gene segments, 25 D gene segments and six J gene segments, and the kappa light chain locus comprises approximately 40 functional V gene segments and five J gene segments. Lucas (2003) Encyclopedia of Life Science 1-8. Combinatorial assortment of these germline genetic elements leads to baseline level of antibody sequence diversity, which is augmented by junction mutations (e.g. deletions, and N- and P-nucleotide additions) and somatic hypermutation to generate a bewildering array of antibody sequence diversity. This sequence diversity is advantageous in providing antibodies to any number of potential pathogens, but it introduces complexity in the isolation and purification of these pools of polyclonal antibodies. One seeking individual antibodies with superior properties for a given purpose will want to screen a large population of antibodies with the greatest possible sequence diversity. This requires not only generating a diverse pool of polyclonal antibodies, e.g. within a transgenic animal, but also recovery of the full breadth of the population of antibodies produced by the animal. The antibody recovery process may inadvertently introduce selection steps in which certain sequences are preferentially retained or lost, reducing the sequence diversity in the recovered polyclonal antibody pool. Ideally, the system for generating and recovering pools of polyclonal antibodies will be designed from the outset to facilitate unbiased generation and recovery of pools of polyclonal antibodies for use in subsequent selection steps.

Transgenic animals, such as mice, have been engineered to express human germline immunoglobulin genes for generation of human antibody antigen-binding domains for use as therapeutics. Such animals comprise a plurality of human leader/V gene segments, as well as D and J gene segments, to enable combinatorial diversity. Such mice may optionally express these human variable domain loci in combination of one or more constant domain sequences native to the host animal, such a mouse, and optionally at the endogenous mouse locus, to enhance efficient somatic hypermutation, and thus affinity maturation, of the antibodies during development in the host animal. Murphy et al. (2014) Proc. Nat'l Acad. Sci. (USA) 111:5153. The animals are injected with an antigen of interest one or more times according to an immunization protocol to elicit production of anti-antigen antibodies. The resulting polyclonal population of antibodies is then recovered, e.g., by fusion of spleen cells from the immunized mice with myeloma cells to form hybridoma cells from which antibodies are isolated and studied. The fusion process and the antibody production steps, however, may constitute de facto selection steps that enrich the resulting pool of antibodies for some sequences at the expense of others, leading to a less diverse pool of antibodies.

Alternatively, B cells from the immunized mice may be separated and individually sequenced to obtain antibody heavy and light chain sequences for direct cloning in a process known as single B cell cloning. See, e.g., Tiller et al. (2008) J. Immunol. Meth. 329:112; Wardemann & Busse (2019) Expression Cloning of Antibodies from Single Human B Cells. In: Küppers R. (eds) Lymphoma. Methods in Molecular Biology, vol. 1956. Humana Press, New York, N.Y. Single B cell antibody cloning and sequencing minimizes the number of steps between the full antibody diversity in the immunized animal and the final pool of polyclonal antibodies by directly interrogating the population of B cells in the animal.

In either case antibody variable domain sequences are obtained by polymerase chain reaction (PCR) using primers hybridizing to sequences 5′ and 3′ of the heavy and light chain variable domains. PCR primers must be designed to accommodate the fact that the sequences flanking the variable domain differ between antibodies in the pool due to different V gene usage and isotype. If one is primarily interested in only IgG antibodies, the sequence 3′ (reverse) primer may be, for example, based on conserved sequence at the 5′ end of the CH1 domain of all IgG constant domains. But the primer at the 5′ end must accommodate antibodies derived from any of the approximately 45 leader/V gene segments, which have correspondingly diverse leader peptide-encoding sequences.

One approach is to use a mixture of primers, each of which anneals to the leader peptide-encoding sequence naturally associated with a different V gene segment, as illustrated in FIG. 1. FIG. 1 provides a schematic representation of a human immunoglobulin heavy chain variable domain locus as typically found in current transgenic animals used to generate human antibodies. FIG. 1 does not represent the present invention. An array of gene segments for a heavy chain variable locus in the animal genome is shown, wherein each leader/V gene segments includes a distinct leader peptide-encoding sequence associated with that specific V gene segment. FIG. 1 further provides schematic representations of two PCR reactions used to recover variable region sequences of fully rearranged antibodies from B cells of immunized animals. In PCR #1, a mixture of forward primers (primer 1-for) and a reverse primer (primer 1-rev) are used to amplify the variable region. A mixture of primers is required for use as the forward primer because the leader peptide-encoding sequences for leader/V gene segments differ. Diversity in V gene segment use is a critical factor in diversifying the potential binding epitopes, specificities and affinities of a population of polyclonal antibodies. Primer 1-for hybridizes to the 5′ end of the leader peptide-encoding sequence of the leader/V gene segment in question, which is a known sequence for each individual leader/V gene segment, thus amplifying the entire leader/V gene segment including the leader peptide-encoding sequence. Primer 1-rev hybridizes to a sequence in the 5′ region of the CH1 domain that is conserved between all IgG isotypes (IgG1, IgG2, IgG3, IgG4).

A second (nested) PCR step (PCR #2) is then performed on the product of PCR #1. Like PCR #1, PCR #2 uses a mixture of forward primers (primer 2-for) and a reverse primer (primer 2-rev), or at most a mixture of a few reverse primers, each primer having a 5′ “tail” region to add additional sequence just outside the variable region. Primer 2-for must be a mixture of primers for the same reason that primer 1-for was, i.e. the sequence it hybridizes to (FR1) differs between V gene segments, so it is necessary to include primers for all possible germline FR1 sequences to ensure amplification of the full diversity of antibody sequences present. Primer 2-rev hybridizes to framework region 4 (FR4) of the variable region, which is somewhat conserved among the human V gene segments and thus primer 2-rev may be a single sequence or a small mixture of sequences. The product of PCR #2 is the variable region with an artificial 5′ “tail” instead of the leader peptide-encoding sequence, and an artificial 3′ “tail” in place of the constant domain/Fc sequence. These 5′ and 3′ tails are designed to anneal with upstream and downstream constructs comprising an optimal promoter and leader peptide-encoding sequence, and a desired Fc sequence, respectively, as shown.

The use of mixtures of primers for primer 1-for and primer 2-for adds complexity, expense, and can hinder amplification efficiency and specificity. A mixture of primers necessarily requires synthesis of many different oligonucleotides to ensure efficient amplification of a full complement of human leader/V gene segments. The relative concentrations of these primers must then be optimized to ensure equivalent amplification efficiencies for the various different leader peptide-encoding sequences, so as to avoid bias in amplification which could lead to loss of less efficiently amplified sequences.

Use of a mixture of primers can also lead to inferior amplification. Primer 1-for, for example, is used for PCR #1, the initial amplification of the variable region from a complex mixture comprising all nucleic acid sequences of the host cell from which the antibody sequence is to be obtained. The presence of a large number of different primer sequences in primer 1-for increases the chances of spurious amplification products arising from priming at genomic DNA sequences that just happen to be complementary to, or nearly complementary to, a primer. PCR #2 is performed on a relatively purified nucleic acid, i.e. the product of PCR #1, but nonetheless suffers from the same expense and optimization issues as PCR #1.

FIG. 2 provides a schematic representation of one embodiment of a new and improved set of genetic elements for use in transgenic animals used to generate human antibodies. The array of gene segments differs from that in FIG. 1 in that all leader/V gene segments are the same color (black) because each leader/V gene segment includes the same leader peptide-encoding sequence. Because all leader/V gene segments have the same leader peptide-encoding sequence, only a single primer sequence is needed for primer 1-for and primer 2-for in PCR #1 and PCR #2, respectively. This obviates the need for complex mixtures of primers and ensures uniform amplification of all V gene segments, and thus maximal diversity of sequence in the polyclonal pool of anti-antigen antibodies obtained.

As illustrated in FIG. 2, the product of PCR #2 includes the leader peptide-encoding sequence and variable region sequence with an artificial 5′ extension, and an artificial 3′ extension. These 5′ and 3′ extensions are designed to anneal with upstream and downstream constructs comprising an optimal promoter sequence, and a desired Fc sequence, respectively. Such genetic constructs are the building blocks for the polynucleotides and transgenic animals of the present invention, the central feature of which is a common leader peptide-encoding sequence associated with each V gene segment. The specific details of the exemplary PCR protocol of FIG. 2 do not limit the present invention, which may be performed by any PCR amplification method suitable to take advantage of the presence of the common leader peptide-encoding sequence associated with each V gene segment.

The use of a common leader peptide sequence for all antibodies in a pool of polyclonal antibodies may provide additional advantages with regard to uniformity across different V gene segment sequences for all aspects of antibody generation. The common germline leader peptide-coding sequence may increase uniformity of genetic rearrangement among V gene segments. The uniform sequence for the leader peptide-encoding portion of the mRNA may enhance uniformity of translation, and thus antibody expression, for example during affinity maturation within the host cell and in transient expression in vitro for screening. The uniform signal peptide on nascent antibody chains may increase uniformity of protein processing and secretion.

Advantages arising solely from use of a uniform polypeptide sequence may be achieved regardless of the DNA sequence encoding it, and regardless of whether the DNA sequences encoding the leader peptide are the same among all or substantially all leader/V gene segments. The additional advantages arising from the use of a uniform DNA sequence, such as the ability to use a single upstream primer in PCR reactions, however, require that the same DNA sequence be used to encode the leader peptide in all or substantially all leader/V gene segments.

Although the benefits of the present invention arise primarily from the efficiencies of having a common leader peptide-encoding sequence for a plurality of human V gene segments, additional human leader/V gene segments that do not share the same leader peptide-encoding sequence may also be present in the transgenic non-human animal. Such additional V gene segments would not necessarily interfere with the benefits arising from the use of a common leader peptide-encoding sequences for the other V gene segments. Nonetheless, in most embodiments there are no human leader/V gene segments other than those comprising the common leader peptide-encoding sequence.

### Leader Peptides

Leader peptides, also referred to as signal peptides, are short stretches of ˜12-30 amino acids at the N-terminus of secreted proteins that target the nascent polypeptide chain for the endoplasmic reticulum. Leader peptides are cleaved from a proprotein to create a mature protein during the process of secretion, and thus these amino acid sequences do not impact the activity of the protein after secretion. Leader peptides are generally diverse in sequence, with a highly diverse N-terminal region, and central region of 7-15 hydrophobic residues, followed by a stretch of about 2-9 small polar residues making up a motif that is cleaved by signal peptidase. Holden et al. (2005) J. Biol. Chem. 280:17172.

Human leader peptide sequences and leader peptide-encoding sequences, such as those associated with human heavy and light chain V gene segments, are available in public databases, are known in the art, and are exemplified at Tables 2 and 3, and the Sequence Listing. Leader peptide-encoding sequences associated with commonly used human V gene segments may be selected because they are known to function well in the context of human antibody expression, especially if subsequent production of the selected antibody in human cells is contemplated. A number of such human V gene leader peptide-encoding sequences, for both heavy and light chains, are provided at Tables 2 and 3.

Alternatively, a leader peptide sequence from the transgenic host animal in which the antibody is to be raised, such as a mouse, may be selected in the expectation that such a leader peptide will function in the animal during antibody development, such as during affinity maturation. An exemplary mouse immunoglobulin gene leader peptide sequence is provided at Table 4.

In addition, human non-immunoglobulin protein leader peptides, leader peptides from other species and artificial/synthetic leader peptides may be used to take advantage of efficient leader peptide sequences regardless of their origin. For example, an artificial leader peptide sequence, referred to as secrecon (SEQ ID NOs: 61 and 124), was created based on computational modeling. Barash et al. (2002) Biochem. Biophys. Res. Commun. 294:835. Another leader sequence, Gaussia luciferase leader peptide (SEQ ID NOs: 67 and 130), was obtained from luciferase produced by Gaussia princeps. WO 2017/068142. Leader peptide and leader peptide-encoding sequences from silk worms, viruses, and various human non-immunoglobulin genes are provided at Table 4 and in the Sequence Listing.

In one aspect, the present invention is independent of the specific choice of leader peptide, and leader peptide-encoding sequence, and is instead simply based on the use of the same leader peptide-encoding sequence for all V gene segments. In another aspect, a specific leader peptide-encoding sequence is selected for use in the polynucleotides and transgenic animals of the invention based on desirable properties, such as efficient antibody gene amplification and subsequent antibody production in a desired expression system, such as the transgenic animal in which it is raised for a cell line, for example human embryonic kidney (HEK) cells or Chinese hamster ovary (CHO) cells.

Exemplary leader peptide-encoding sequences for use in the various embodiments of the present invention are described below at Tables 2, 3 and 4, and are provided in the Sequence Listing submitted herewith, which is hereby incorporated by reference in its entirety. Exemplary leader peptide-encoding sequences are provided at SEQ ID NOs: 1-70, 134, 136 and 137, with the corresponding amino acid sequences at SEQ ID NOs: 71-133 and 135. These Tables provide names of genes from which the leader sequences were obtained or derived, and sequence identifier numbers for the leader peptide amino acid sequence and encoding DNA sequence. Tables 2 and 3 also provide sequence reference numbers for obtaining corresponding human genomic sequences from the ImMunoGeneTics (IMGT®) immunoglobulin sequence database. Genomic sequence are provided in Table 2 for selected leader peptide encoding sequence, i.e. IGHV 3-23 (SEQ ID NO: 136) and IGKV 3-20 (SEQ ID NO: 137), that include not only the coding sequence for the leader peptide but also the intron associated with the specific human leader peptide encoding sequence. Inclusion of such intron-containing genomic sequence, rather than just the coding sequence, may have advantages when included in the transgenic mice of the present invention.

TABLE 2
Exemplary Heavy Chain V Gene-Derived Leader Peptide Sequences

| | Coding | IMGT ® | Amino Acid |
|---|---|---|---|
| | Sequence | Genomic | Sequence |
| | (SEQ | Sequence | (SEQ |
| Gene | ID NO:) | Reference | ID NO:) |
| IGHV 1-2 | 1 | X07448 | 71 |
| IGHV 1-3 | 2 | X62109 | 72 |
| IGHV 1-18 | 3 | M99641 | 73 |
| IGHV 1-24 | 4 | AB019439 | 74 |
| IGHV 1-45 | 5 | X92209 | 75 |
| IGHV 1-46 | 6 | X92343 | 76 |
| IGHV 1-58 | 7 | M29809 | 77 |
| IGHV 1-69 | 8 | L22582 | 78 |
| IGHV 2-26 | 9 | M99648 | 79 |
| IGHV 2-70 | 10 | L21969 | 80 |
| IGHV 3-7 | 11 | M99649 | 81 |
| IGHV 3-11 | 12 | M99652 | 82 |
| IGHV 3-15 | 13 | X92216 | 83 |
| IGHV 3-20 | 14 | M99657 | 84 |
| IGHV 3-21 | 15 | AB019439 | 85 |
| IGHV 3-23 | 16 | AC245166 | 86 |
| IGHV 3-23-L8V | 17 | based on AC245166 | 84 |
| IGHV 3-23-C19S | 18 | based on AC245166 | 87 |
| IGHV 3-23 genomic | 136 | AC245166 | 86 |
| IGHV 3-30 | 19 | M83134 | 88 |
| IGHV 3-30.3 | 19 | AC244456 | 88 |
| IGHV 3-33 | 19 | AB019439 | 88 |
| IGHV 3-48 | 20 | M99675 | 89 |
| IGHV 3-49 | 21 | M99676 | 84 |
| IGHV 3-53 | 22 | M99679 | 90 |
| IGHV 3-64 | 23 | M99682 | 91 |
| IGHV 3-66 | 14 | X92218 | 84 |
| IGHV 3-72 | 24 | X92206 | 92 |
| IGHV 3-73 | 14 | X70197 | 84 |
| IGHV 3-74 | 14 | L33851 | 84 |
| IGHV 4-28 | 25 | X05714 | 93 |
| IGHV 4-31 | 25 | L10098 | 93 |
| IGHV 4-34 | 26 | AB019439 | 93 |
| IGHV 4-39 | 27 | AB019439 | 93 |
| IGHV 4-39 Long | 134 | AB019439 | 135 |
| IGHV 4-59 | 28 | AB019438 | 93 |
| IGHV 4-61 | 26 | M29811 | 93 |
| IGHV 5-51 | 29 | M99686 | 94 |
| IGHV 6-1 | 30 | J04097 | 95 |

TABLE 3
Exemplary Light Chain V Gene-Derived Leader Peptide Sequences

| | Coding | IMGT ® | Amino Acid |
|---|---|---|---|
| | Sequence | Genomic | Sequence |
| | (SEQ | Sequence | (SEQ |
| Gene | ID NO:) | Reference | ID NO:) |
| IGKV 1-5 | 31 | Z00001 | 96 |
| IGKV 1-6 | 32 | M64858 | 97 |
| IGKV 1-8 | 33 | Z00014 | 98 |
| IGKV 1-9 | 32 | Z00013 | 97 |
| IGKV 1-12 | 34 | V01577 | 99 |
| IGKV 1-16 | 35 | J00248 | 100 |
| IGKV 1-17 | 36 | X72808 | 101 |
| IGKV 1-27 | 37 | X63398 | 102 |
| IGKV 1-33 | 38 | M64856 | 103 |
| IGKV 1-39 | 39 | X59315 | 104 |
| IGKV 1D-8 | 32 | Z00008 | 97 |
| IGKV 1D-13 | 40 | X17262 | 97 |
| IGKV 1D-37 | 41 | X71893 | 105 |
| IGKV 1D-43 | 42 | X72817 | 106 |
| IGKV 2-24 | 43 | X12684 | 107 |
| IGKV 2-28 | 44 | X63397 | 108 |
| IGKV 2-29 | 45 | X63396 | 109 |
| IGKV 2-30 | 46 | X63403 | 110 |
| IGKV 3-11 | 47 | X01668 | 111 |
| IGKV 3-15 | 48 | M23090 | 111 |
| IGKV 3-20 | 49 | X12686 | 112 |
| IGKV 3-20 genomic | 137 | X12686 | 112 |
| IGKV 3D-7 | 50 | X72820 | 113 |
| IGKV 4-1 | 51 | Z00023 | 114 |
| IGKV 5-2 | 52 | X02485 | 115 |
| IGKV 6-21 | 53 | X63399 | 116 |

TABLE 4
Other Leader Peptide Sequences

| | Coding | Amino Acid |
|---|---|---|
| | Sequence | Sequence |
| | (SEQ | (SEQ |
| Gene | ID NO:) | ID NO:) |
| Osteonectin | 54 | 117 |
| H7 | 55 | 118 |
| oncostatin M | 56 | 119 |
| vesicular stomatitis virus-G | 57 | 120 |
| mouse Ig Kappa | 58 | 121 |
| IgG2 | 59 | 122 |
| silkworm (bombyx mori) fibroin LC | 60 | 123 |
| secrecon | 61 | 124 |
| CD33 | 62 | 125 |
| tPA (tissue plasminogen activator) | 63 | 126 |
| chymotrypsinogen B2 | 64 | 127 |
| trypsinogen-2 | 65 | 128 |
| IL-2 | 66 | 129 |
| Gaussia luciferase | 67 | 130 |
| serum albumin | 68 | 131 |
| influenza haemagglutinin | 69 | 132 |
| insulin | 70 | 133 |

### V Gene Segments

Human heavy chain leader/V gene segments are known in the art, and are available, e.g., at Genbank Accession numbers AB019437-AB019441. Matsuda et al. (1998) J. Exp. Med. 188:2151. See also LeFranc (2001) Exp. Clin. Immunogenet. 18:100. A subset of human heavy chain V gene segments of particular interest consists of IGHV genes 1-2, 1-3, 1-18, 1-46, 1-69, 2-5, 2-26, 3-7, 3-9, 3-11, 3-21, 3-23, 3-30, 3-33, 3-48, 3-66, 3-72, 3-74, 4-4, 4-28, 4-31, 4-30-4, 4-34, 4-39, 4-59, 4-61, 5-51 and 7-4-1. These and human heavy chain D gene segments (IGHD), J gene segments (IGHJ) and constant region gene segments (IGHC) for use in the polynucleotides, methods and transgenic animals of the present invention may be obtained from public sequence databases using accession numbers as provided at LeFranc (2001) Exp. Clin. Immunogenet. 18:100. See also OMIM #147070 (Immunoglobulin Heavy Chain Variable Gene Cluster; IGHV) for a description of variable region V, D and J gene segments, and OMIM #147100 (IgG Heavy Chain Locus; IGHG1) for a description of exemplary heavy chain C gene segments (IgG).

Human light chain kappa leader/V gene segments are also known in the art, and are available in public databases, such as Genbank. The kappa light chain locus is on human chromosome 2 at 2p12 (genomic coordinates 2:74,800,000-83,100,000). Human light chain kappa V gene segments (IGKV) of interest include the approximately 40 naturally occurring kappa V gene segments disclosed at LeFranc (2001) Exp. Clin. Immunogenet. 18:161. A subset of human light chain kappa V gene segments of particular interest consists of IGKV genes 1-5, 1-9, 1-12, 1-16, 1-17, 1-27, 1-33, 1-39, 1D-13, 1D-43, 2-28, 2-29, 2-30, 3-11, 3-15, 3-20, 3D-7, 4-1 and 6-21. These and human light chain kappa J (IGKJ) segments and constant region gene segments (IGKC) for use in the nucleic acids, methods and transgenic animals of the present invention may be obtained from public sequence databases using accession numbers as provided at LeFranc (2001) Exp. Clin. Immunogenet. 18:161. See also OMIM #146980 (Immunoglobulin Kappa Light Chain Variable Gene Cluster; IGKV) for a description of variable region kappa V and J gene segments, and OMIM #147200 (Immunoglobulin Kappa Light Chain Constant Region; IGKC) for a description of kappa C gene segments.

The sequences of human V genes are readily available to those of skill in the art in public databases, such as Genbank and especially the ImMunoGeneTics (IMGT®) immunoglobulin sequence database. Lefranc, M.-P. et al. (1999) Nucleic Acids Res., 27:209-212; Ruiz, M. et al. (2000) Nucleic Acids Res., 28:219-221; Lefranc, M.-P. (2001) Nucleic Acids Res., 29:207-209; Lefranc, M.-P., Nucleic Acids Res. (2003) 31:307-310; Lefranc, M.-P. et al. (2004) In Silico Biol., 5, 0006 [Epub], 5:45-60 (2005); Lefranc, M.-P. et al. (2005) Nucleic Acids Res., 33:D593-597; Lefranc, M.-P. et al. (2009) Nucleic Acids Res., 37:D1006-1012; Lefranc, M.-P. et al. (2015) Nucleic Acids Res., 43:D413-422 (2015). Such databases will enable those of skill in the art to obtain coding sequences for all human V gene segments and their naturally associated leader peptide-encoding sequences. In addition to the genomic sequences including introns, annotations for each V gene segment will enable separation of leader peptide-encoding sequence from adjacent framework and CDR-encoding sequences, and will enable construction of simple coding sequences (lacking introns) is such sequences are desired. In one embodiment, the present invention makes use of human V gene associated leader peptide-encoding sequences that retain one or more introns from the human germline sequence, such as the intron typically found in the sequence encoding the C-terminal end of the leader peptide sequence.

### Sequence Variants

In some embodiments in which genetic elements are derived from, or based on, human genes, the leader peptide-encoding sequences and/or the V gene segment sequences comprise the full human genomic sequence including introns. In alternative embodiments in which genetic elements are derived from, or based on, human genes, the genomic leader peptide-encoding sequences and/or the V gene segment sequences comprise only nucleic acid sequences encoding polypeptides, and thus correspond to the sequence of a naturally-spliced mRNA (or the corresponding cDNA). Use of such intronless nucleic acid constructs has the advantage of reducing the size of the genetic elements to be used in constructing the variable region locus of the present invention.

In addition to the naturally occurring genomic sequence for a leader peptide-encoding sequence, and the natural splice product of that sequence, nucleic acids of the current invention also include codon-optimized DNA sequences encoding the leader peptide. It is known that different organisms and different cells preferentially use certain codons encoding a given amino acid residue. Athey et al. (2017) BMC Bioinform.18:391. Codon-optimized sequences are nucleic acid sequences in which codons are modified to optimize expression of the protein, in this case the leader peptide, in the cell in which it will be expressed, such as human cells of Chinese hamster ovary (CHO) cells. See, e.g., Mauro (2018) BioDrugs 32:69. Such codon-optimized nucleic acid constructs have the advantage of improving the efficiency of translation of the heavy or light chain while retaining the function of the leader peptide sequence in the heavy or light chain polypeptide, since the amino acid sequence is unchanged. The leader peptide-encoding sequence for osteonectin leader (SEQ ID NO: 54) is an example of such a codon-optimized sequence, and has seven base pair changes compared with the genomic sequence found at NG_042174.1 residues 10728-10778. Experiments must be performed to determine that such codon-optimization does not interfere with genetic rearrangements needed to generate functional antibody chains, or give rise to unacceptably poor amplification during the process of sequencing the antibody variable regions.

### Methods of Making Transgenic Animals with Improved Human Immunoglobulin Loci for Generation of a Diverse Pool of Polyclonal Antibodies

Transgenic animals of the present invention, comprising in their genomes human heavy and light chain immunoglobulin variable domain loci in which all leader/V gene segments for a particular chain comprise a common, identical, leader peptide-encoding sequence, are created essentially as follows. Briefly, following selection of a single or set of leaders, germline sequences for IGHV and IGKV genes are selected based on functional annotation to include promoters, 5′ UTR, coding, recombination sequences, and flanking germline sequence. Leader sequences are exchanged within the context of germline sequence without disruption of the upstream or downstream sequence functionality. Variable regions are generated by synthesis and assembled into a single construct using standard molecular biology techniques that include but are not limited to: recombineering, golden gate assembly, and restriction enzyme-based ligation generating a variable domain array. The array is then assembled onto additional synthetic or germline IGH or IGK sequence by the aforementioned techniques to generate a targeting vector. The targeting vector includes positive drug selection, homology arms, and/or recombination sequences and is electroporated into embryonic stem cells along with recombinase or nuclease constructs. Drug selection is performed using standard procedure and individual clones are screened by internal and external PCR, TLA Sequencing, or genome wide sequencing to confirm site specific integration. Positive clones are further screened for a 40XY g-band karyotype analysis and chimeras are injected by standard blastocyst injection and transferred to pseudopregnant females. Pups are genotyped to confirm integration by any of the following techniques: PCR, southern blot, TLA Sequencing, or genome wide sequencing to confirm integration. Progeny are maintained at heterozygous or homozygous and intercrossed with relevant alleles for downstream use.

### Transgenic Animals with Improved Human Immunoglobulin Loci for Generation of a Diverse Pool of Polyclonal Antibodies

In another aspect, the invention provides transgenic animals, such as transgenic mice, comprising in their genome a human heavy and light chain variable region loci comprising a plurality of different leader/V gene segments all sharing a common leader peptide-encoding sequence for a given chain. Such animals may also comprise D gene segments (for heavy chains) and J gene segments capable of rearrangement with the leader/V gene segments to form rearranged heavy and light chain variable regions. Human variable region gene segments in the transgenic animals of the present invention are typically naturally occurring sequences, including allotypic variants, but engineered sequences may also be used. The human variable region gene segments may be located at the locus of the corresponding non-human variable region gene segments, or may be located at an exogenous locus. Transgenic animals of the present invention may comprise the full repertoire of human variable region gene segments, such as the full repertoire of human V gene segments, or a subset of such segments.

Such animals may further comprise constant region gene segments capable of rearrangement with the variable regions to form full-length antibody heavy chains and light chains. In some embodiments the constant region gene segments are of human germline original, and in other embodiments the constant region gene segments are native to the transgenic animal, e.g. a mouse comprising mouse constant region gene segments. Transgenic animals comprising fully humanized immunoglobulin loci have the advantage of producing fully human antibodies directly, which antibodies may be selected for use as human therapeutics without sequence modification. Transgenic animals comprising a humanized variable region locus and an endogenous constant region produce chimeric antibodies that have to be modified to introduce a human constant region before being suitable for use as human therapeutics, but have the advantage that the endogenous constant region on the antibodies can direct more efficient class-switching and affinity maturation during the immune response in the transgenic animal. Murphy et al. (2014) Proc. Nat'l Acad. Sci. (USA) 111:5153.

Transgenic animals of the present invention may comprise the full repertoire of human constant region gene segments, or a subset of such segments, such as only IgG constant region gene segments. Human constant region gene segments of the transgenic animals of the present invention are typically naturally occurring sequences, including allotypic variants, but engineered sequences may also be used, such as variants engineered to increase or decrease binding to certain Fcγ receptors, such activating Fcγ receptors.

The transgenic animals of the present invention may optionally comprise, in addition to the human variable region locus, endogenous immunoglobulin variable region gene segments of the transgenic host animal. Such non-human gene segments may be in their native orientation in the transgenic animal, preferentially inactivated or silent, or may be inverted so as to impair their function. Lee et al. (2014) Nat. Biotechnol. 32:356.

### Methods of Generating Antibodies Using the Transgenic Animals of the Present Invention

In another aspect, the invention provides methods of generating antibodies using the transgenic animals of the present invention. Transgenic animals of the present invention, comprising a human heavy and/or light chain variable region immunoglobulin loci with a plurality of heavy and/or light chain leader/V gene segments having identical leader peptide-encoding sequences, can be immunized with an antigen of interest according to an immunization protocol as is known in the art. Chen & Murawsky (2018) Front. Immunol. 9:460; Asensio et al. (2019) mAbs 11:870. For example, a protein antigen can be presented as a soluble protein, as peptide fragment, expressed on the surface of a cell, as a DNA expression construct, or a series or combination of these. Antigen may be presented in one or a series of administrations, e.g. under a prime and boost protocol, such as multiple injections per week for four weeks, or one injection every four weeks for 12 weeks. Antigen may be administered, for example, subcutaneously (e.g. into a mouse footpad or base of the tail) or intraperitoneally. Antigen may be administered with or without an adjuvant, such as alum, Complete Freund's Adjuvant (CFA), Seppic Montanide ISA50, or alhydrogel/muramyl dipeptide (ALD/MDP).

After an appropriate interval for antibody titer to rise and affinity maturation, antibody sequences are obtained from the mice by conventional means, such as hybridoma formation and sequencing of the heavy and light chain variable domains for clones producing antibodies with desirable properties, or single B cell antibody cloning and sequencing.

If an antibody is intended for therapeutic use in humans, and was made in an animal, such as a mouse, that produces chimeric antibodies having animal-derived constant domain sequences, then the human variable domain is reformatted into a construct providing a human constant domain sequence.

### Antibodies Made Using the Transgenic Animals of the Present Invention

In another aspect, the invention provides antibodies made using the methods of generating the transgenic animals of the present invention. Human antibodies of the present invention may be obtained from transgenic animals with immunoglobulin loci humanized at both the variable region and the constant region, directly giving a fully human antibody, or in a transgenic animal humanized only at the variable region, giving a chimeric human/animal antibody for which the constant region may be replaced with a human constant region if it is to be used as a human therapeutic, using methods known in the art.

Antibodies obtained by immunization of the transgenic animals of the present invention will initially comprise a pool of polyclonal antibodies of different sequences, from which individual antibodies may be selected for desired properties. Polyclonal antibody pools of the present invention will comprise individual antibodies derived from a greater diversity of V gene segments, and/or derived more uniformly from any given set of different V gene segments, than antibody pools obtained from conventional transgenic humanized immunoglobulin mice having the native leader peptide-encoding sequences associated with each V gene segment. Antibodies may be selected for any number of desired properties, or combination of properties, depending on the intended use of the antibodies.

Increased V gene usage and recovery provides increased sequence diversity, allowing selection of antibodies without sequence liabilities that can adversely influence stability, developability, and production yield. For example, antibody stability and homogeneity may be improved by selecting antibodies lacking sequences known to be susceptible to glycosylation (e.g. N-x-S/T and N-x-C), deamidation (e.g. NG and NS motifs), isomerization (e.g. DG and DS motifs), and oxidation (e.g. W, F, M or C residues). Lu et al. (2019) MAbs 11:45. Although such sequence liabilities may be engineered out of antibodies rather than selected, such sequence modifications may interfere with antigen binding and must be tested.

Increased V gene usage may also result in improved epitope diversity, i.e. an increase in the variety of different loci on the antigen for which antibodies are found. Increased epitope diversity may allow for selection of antibodies with improved properties, including but not limited to, high affinity, pH sensitive binding, species cross-reactivity, cross-reactivity to related antigen sequences, specificity for blocking of a specific binding partner, ability to block the binding of two or more binding partners, ability to bind without blocking the binding of one or more binding partners (so-called “non-blocking” antibodies), binding to antigen when expressed on a cell surface, binding to an antigen on a cell surface without triggering transmembrane signaling, binding to an antigen on a cell surface while triggering maximal transmembrane signaling, binding to antigen simultaneously with a second anti-antigen antibody, binding to denatured antigen, binding to antigen in tissue sections, etc. For example, cross-reactivity to human and animal antigen will be useful in therapeutic antibodies because the antibody may be used directly in toxicology models in that animal. Non-blocking antibodies can be used in therapeutic applications when the therapeutic mechanism does not require, or precludes, reduced binding to a binding partner. Antibodies that can bind to antigen on the surface of a cell may be necessary for therapeutic methods requiring such binding, such as delivery of a cytotoxic payload or transmembrane signaling. Antibodies that can bind to denatured antigen, antigen deposited on a plate, or that can bind at the same time as other anti-antigen antibodies may find use in various assays such as ELISA, immunohistochemistry (IHC) and flow cytometry. Antibodies that specifically block just one interaction, or antibodies that block two or more interactions, may find use in therapeutic contexts where the specific pattern of blocking is mechanistically preferred.

### Methods of Treatment Using Antibodies Made Using the Transgenic Animals of the Present Invention

In another aspect, the invention provides methods of treatment, e.g. of human disease, using therapeutic antibodies obtained using the transgenic animals of the present invention. Antibodies of the present invention that have been raised against therapeutic targets may be used to treat corresponding diseases associated with that target. For example, inflammatory cytokines may be used to generate antagonist antibodies that can be used to treat autoimmune and inflammatory disorders. See, e.g., Singh et al. (2018) Curr. Clin. Pharmacol. 13:85. Such targets include but are not limited to IL-1β, IL-2, IL-4, IL-5, IL-6R, IL-13, IL-12 (p40 subunit), IL-17, IL-23 (p19 subunit), TNF-α or any of their receptors. Immuno-oncology targets, which are typically cell surface receptors involved in mediation of immune response to tumors, may be used to generate antibodies that can be used to treat cancers. Such targets include but are not limited to CTLA-4, PD-1, PD-L1, LAG3, TIM-3, TIGIT, ICOS, CD27, KIRm 4-1BB (CD137), OX40 (CD134) and CD96. Tumor antigens and tumor-specific cell surface markers may also be used to generate antibodies that can be used to treat cancer. Such targets include but are not limited to HER-2, EGFR, VEGF, VEGFR2, Fucosyl-GM1, mesothelin, CD19, CD20, CD30, CD33, CD38, CD52 and SLAMF7.

Examples of cancers that may be treated using the immunotherapeutic methods of the disclosure include bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, a hematological malignancy, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, metastatic cancers, and any combinations of said cancers.

Other cancers include hematologic malignancies including, for example, multiple myeloma, B-cell lymphoma, Hodgkin lymphoma/primary mediastinal B-cell lymphoma, non-Hodgkin's lymphomas, acute myeloid lymphoma, chronic myelogenous leukemia, chronic lymphoid leukemia, follicular lymphoma, diffuse large B-cell lymphoma, Burkitt's lymphoma, immunoblastic large cell lymphoma, precursor B-lymphoblastic lymphoma, mantle cell lymphoma, acute lymphoblastic leukemia, mycosis fungoides, anaplastic large cell lymphoma, T-cell lymphoma, and precursor T-lymphoblastic lymphoma, and any combinations of said cancers.

### Polynucleotides Comprising a Plurality of Human V Gene Segments Preceded by Identical Leader Peptide-Encoding Sequences

In another aspect, the invention provides polynucleotides, typically DNA constructs, comprising a plurality (i.e. two or more) of human heavy or light chain leader/V gene segments in which each leader/V gene segment comprises an identical leader peptide-encoding sequence. Such polynucleotides may be incorporated into larger polynucleotides further comprising one or more D gene segments (for heavy chain variable region constructs) and one or more J gene segments. Heavy chain variable region loci may be incorporated into yet larger polynucleotides further comprising one or more human constant region gene segments, such as human IgG gene segments, such as human IgG1, IgG2, IgG3 or IgG4. Light chain variable region loci may be incorporated into yet larger polynucleotides further comprising one or more human constant region gene segments, such as human κ or λ light chain constant region sequences.

The present invention is further illustrated by the following examples, which should not be construed as limiting. The contents of all figures and all references, patents and published patent applications cited herein are expressly incorporated herein by reference.

### EXAMPLE 1

### Selection of Optimal Leader Peptide-Encoding Sequences

Selection of optimal leader peptide sequences, and optimal leader peptide-encoding sequences, for use in the methods, constructs and animals of the present invention, were selected as follows. Briefly, an initial panel of potential leader peptide sequences was selected based on conservation and consensus of the sequence within the group of all leader sequences, frequency and usage in human antibody repertoire, and to maximize sequence diversity across the initial panel. Both amino acid and DNA sequences were also ranked based on their prevalence in human antibody repertoires generally, and in approved antibody drugs, on the basis that such selections suggest sequences that are biologically desirable, amenable to commercial antibody production cell lines and methods, and for use in human therapeutics. This panel of leader peptides was then evaluated for efficiency of recombinant protein expression by pairwise evaluation of heavy chain and light chain leader combinations alongside control leaders, including osteonectin (SEQ ID NO: 117), which is frequently used for this purpose. This initial evaluation allowed us to exclude suboptimal leaders and to select leader candidates that would be sufficient for in vitro expression. Expression of two exemplary antibodies was determined using selected pairwise combinations of heavy and light chain leader sequences of the panel to drive expression of the heavy and light chains, respectively. Results showed that although some leaders were very poor, most leaders support adequate expression at or above the level of the osteonectin leader sequence. The benefit of selecting a common leader set that performs well for in vitro expression enables strategic flexibility in using the common leader in recombinant vectors and for direct use in transcriptionally active polymerase chain reaction amplifications of heavy chain and light chain sequences to screen lead antibody candidates directly from the animal, thereby integrating a universal sequence recovery strategy with a downstream functional screening strategy. Benefits include potentially improved expression over osteonectin, simplified molecular biology strategies moving from the animal to the in vitro setting that may improve recovery of sequences, and the ability to scale the process for automation.

The panel of leaders was further refined following initial functional screening by in vitro expression. These criteria included an evaluation of the complexity and length of the genomic sequence to determine if it would accommodate engineering across multiple variable genes for inclusion in transgenic constructs. An exome blast confirmed that the sequences used for primer design would be unique across the mouse transcript and support specificity. Functional liabilities in the recombinant setting were considered as well to further refine criteria for high-value leaders. For instance, leader sequences were also eliminated if they include a downstream methionine residue, such as IGKV 1-9 (SEQ ID NO: 97) and IGKV 1-39 (SEQ ID NO: 104), to avoid its use as a cryptic translation start site.

In addition, the sequences of potential amplification primers for use with the leader peptide-encoding sequences were analyzed for undesirable secondary structure and sequence liabilities that would impact the ability to design high-performing PCR strategies. These included primer sequences immediately adjacent to the variable domain framework near the 3′ region of the leader, which is a strategic region for supporting full-length variable domain sequencing since more 5′ regions would create longer sequences to resolve by sequencing.

Based on the considerations above, the heavy chain leader peptide-encoding sequence for IGHV 3-23 (SEQ ID NOs: 16 and 136), which encode SEQ ID NO: 86, were selected for heavy chains in the methods, constructs and mice of the present invention, as was light chain leader peptide-encoding sequence for IGKV 3-20 (SEQ ID NOs: 49 and 137), which encode SEQ ID NO: 112. In a preferred embodiment the genomic leader peptide-encoding sequences of IGHV 3-23 (SEQ ID NO: 136) and IGKV 3-20 (SEQ ID NO: 137) are used for heavy and light chain V gene segments, respectively. Although these sequences were determined to be optimal based on the criteria presented herein, other sequences may be employed in the methods, constructs and mice of the present invention since use of the same leader peptide-encoding sequence is inherently beneficial regardless of which specific sequence is selected.

### EXAMPLE 2

### Selection of Heavy and Light Chain V Gene Segments

V gene segments for use in the methods and mice of the present invention were selected as follows. Briefly, V gene segments for both heavy and light chains were selected based on their prevalence in human antibody repertoires generally, in antibody therapeutics that have entered Phase I clinical trials, and in approved antibody drugs. They were also assessed for chemical liabilities, such as the presence of methionines (especially in CDRs) or unpaired cysteines, and sequence liabilities like DG and NG, the presence of glycosylation sites, and immunogenicity—both experimentally observed and computationally predicted (e.g. by EPIVAX® immunogenicity assessment software, EpiVax Inc., Providence, R.I., USA).

After consideration and balancing of the factors above, the following 19 V gene segments (hIGHV) were selected for the heavy chain variable domain locus: 3-23; 5-51; 3-7; 1-2; 1-69-1; 3-48; 1-18; 1-46; 3-21; 3-30; 3-74; 4-39; 3-9; 2-5; 1-3; 4-4; 7-4-1; 3-66; and 1-24. The following 17 V gene segments (hIGKV) were selected for the light chain variable domain locus: 1-39; 3-11; 1-33; 3-20; 4-1; 1-27; 1-5; 1-16; 1-12; 2-30; 3-15; 2-28; 1D-13; 1-17; 6-21; 1-9; and 1D-43.

Engineering of the common leaders into IGH and IGK variable genes was addressed by the observation that functional variable domain genes are two exons separated by an intron of variable but moderate length and the germline leader ends in the second exon. This enabled a universal approach to transgene design for all variable genes by including the germline DNA sequence for the common leader from ATG in exon 1 through the end of the common leader in exon 2. A universal strategy ensures that each variable domain gene can be successfully designed without introducing functional liabilities, such as dysfunctional splicing across the intron or negatively impacting expression of the variable domain genes.

The contents of all figures and all references, patents and published patent applications cited throughout this application are expressly incorporated herein by reference.

### Equivalents

Those skilled in the art will recognize, or be able to ascertain using no more than routine experimentation, many equivalents of the specific embodiments disclosed herein. Such equivalents are intended to be encompassed by the following claims.

1. A method of making a non-human transgenic animal having a human immunoglobulin variable region locus, comprising introducing into the genome of a non-human animal a plurality of human heavy chain leader/V gene segments, wherein each of the plurality of human heavy chain leader/V gene segments comprises the same first leader peptide-encoding sequence.

2. The method of claim 1 wherein no additional human heavy chain leader/V gene segments are introduced, or are present in the genome of the non-human animal, other than the plurality of human heavy chain leader/V gene segments comprising the same first leader peptide-encoding sequence.

3. The method of claim 2 further comprising introducing into the genome of a non-human animal a plurality of human light chain leader/V gene segments, wherein each of the human light chain leader/V gene segments comprises the same second leader peptide-encoding sequence.

4. The method of claim 3 wherein no additional human light chain leader/V gene segments are introduced, or are present in the genome of the non-human animal, other than the plurality of human light chain leader/V gene segments comprising the same second leader peptide-encoding sequence.

5-6. (canceled)

7. The method of claim 4 wherein the first leader peptide-encoding sequence is not the same as the second leader peptide-encoding sequence.

8-36. (canceled)

37. A transgenic non-human animal comprising in its genome a plurality of human heavy chain leader/V gene segments, wherein each of the human heavy chain leader/V gene segments comprises the same first leader peptide-encoding sequence.

38. The transgenic non-human animal of claim 37 wherein no additional human heavy chain leader/V gene segments are present in the genome of the non-human transgenic animal other than the plurality of human heavy chain leader/V gene segments comprising the same first leader peptide-encoding sequence.

39. The transgenic non-human animal of claim 38 further comprising in its genome a plurality of human light chain leader/V gene segments, wherein each of the human light chain leader/V gene segments comprises the same second leader peptide-encoding sequence.

40. The transgenic non-human animal of claim 39 wherein no additional human light chain leader/V gene segments are present in the genome of the non-human transgenic animal other than the plurality of human light chain leader/V gene segments comprising the same second leader peptide-encoding sequence.

41. (canceled)

42. The transgenic non-human animal of claim 40 wherein the first leader peptide-encoding sequence is not the same as the second leader peptide-encoding sequence.

43-71. (canceled)

72. A method of manufacturing a human antibody, or antigen binding fragment thereof, comprising:
a. administering an antigen of interest to the transgenic non-human animal of claim 42;
b. obtaining a nucleic acid sequence encoding the antigen binding domains of the heavy and light chains of an antibody specific for the antigen produced by the transgenic non-human animal after step (a);
c. expressing an antibody specific for the antigen, or antigen binding fragment thereof, from a genetic construct comprising one or both of the nucleic acid sequences obtained in step (b); and
d. isolating or purifying the antibody or antigen binding fragment expressed in step (c).

73. The method of claim 72 wherein the non-human transgenic animal is a mouse.

74. A method of treating a subject having a disease, comprising administering to the subject an antibody, or antigen binding fragment thereof, produced by the method of claim 73.

75. The method of treating of claim 74 wherein the subject is a human.

76. (canceled)

77. A polynucleotide comprising two or more human heavy or light chain leader/V gene segments comprising identical leader peptide-encoding sequences.

78. The polynucleotide of claim 77 wherein the two or more human heavy chain leader/V gene segments comprise two or more different naturally occurring human V gene segments, and further wherein each leader/V gene segment comprises the same first leader peptide-encoding sequence, and further wherein said first leader peptide-encoding sequence encodes a leader peptide sequence selected from the group consisting of SEQ ID NOs: 71-133 and 135.

79-80. (canceled)

81. The polynucleotide of claim 77 wherein the two or more human light chain leader/V gene segments comprise two or more different naturally occurring human V gene segments, and further wherein each leader/V gene segment comprises the same second leader peptide-encoding sequence, and further wherein said second leader peptide-encoding sequence encodes a leader peptide sequence selected from the group consisting of SEQ ID NOs: 71-133 and 135.

82-83. (canceled)


SEQUENCE LISTING (137 sequences)

SEQ ID NO: 1 - DNA, 57 bp, Homo sapiens
atggactggacctggaggatcctcttcttggtggcagcagccacaggagcccactcc

SEQ ID NO: 2 - DNA, 57 bp, Homo sapiens
atggactggacctggaggatcctctttttggtggcagcagccacaggtgcccactcc

SEQ ID NO: 3 - DNA, 57 bp, Homo sapiens
atggactggacctggagcatccttttcttggtggcagcagcaacaggtgcccactcc

SEQ ID NO: 4 - DNA, 57 bp, Homo sapiens
atggactgcacctggaggatcctcttcttggtggcagcagctacaggcacccacgcc

SEQ ID NO: 5 - DNA, 57 bp, Homo sapiens
atggactggacctggagaatcctcttcttggtggcagcagtcacagatgcctactcc

SEQ ID NO: 6 - DNA, 57 bp, Homo sapiens
atggactggacctggagggtcttctgcttgctggctgtagctccaggtgctcactcc

SEQ ID NO: 7 - DNA, 57 bp, Homo sapiens
atggactggatttggaggatcctcttcttggtgggagcagcgacaggtgcccactcc

SEQ ID NO: 8 - DNA, 57 bp, Homo sapiens
atggactggacctggaggttcctctttgtggtggcagcagctacaggtgtccagtcc

SEQ ID NO: 9 - DNA, 57 bp, Homo sapiens
atggacacactttgctacacactcctgctgctgaccaccccttcctgggtcttgtcc

SEQ ID NO: 10 - DNA, 57 bp, Homo sapiens
atggacatactttgttccacgctcctgctactgactgtcccgtcctgggtcttatcc

SEQ ID NO: 11 - DNA, 57 bp, Homo sapiens
atggagttggggctgagctgggttttccttgttgctattttagaaggtgtccagtgt

SEQ ID NO: 12 - DNA, 57 bp, Homo sapiens
atggagtttgggctgagctgggttttccttgttgctattataaaaggtgtccagtgt

SEQ ID NO: 13 - DNA, 57 bp, Homo sapiens
atggagtttgggctgagctggattttccttgctgctattttaaaaggtgtccagtgt

SEQ ID NO: 14 - DNA, 57 bp, Homo sapiens
atggagtttgggctgagctgggttttccttgttgctattttaaaaggtgtccagtgt

SEQ ID NO: 15 - DNA, 57 bp, Homo sapiens
atggaactggggctccgctgggttttccttgttgctattttagaaggtgtccagtgt

SEQ ID NO: 16 - DNA, 57 bp, Homo sapiens
atggagtttgggctgagctggctttttcttgtggctattttaaaaggtgtccagtgt

SEQ ID NO: 17 - DNA, 57 bp, Homo sapiens
atggagtttgggctgagctgggtttttcttgtggctattttaaaaggtgtccagtgt

SEQ ID NO: 18 - DNA, 57 bp, Homo sapiens
atggagtttgggctgagctggctttttcttgtggctattttaaaaggtgtccagtcc

SEQ ID NO: 19 - DNA, 57 bp, Homo sapiens
atggagtttgggctgagctgggttttcctcgttgctcttttaagaggtgtccagtgt

SEQ ID NO: 20 - DNA, 57 bp, Homo sapiens
atggagttggggctgtgctgggttttccttgttgctattttagaaggtgtccagtgt

SEQ ID NO: 21 - DNA, 57 bp, Homo sapiens
atggagtttgggcttagctgggttttccttgttgctattttaaaaggtgtccaatgt

SEQ ID NO: 22 - DNA, 57 bp, Homo sapiens
atggagttttggctgagctgggttttccttgttgctatttcaaaaggtgtccagtgt

SEQ ID NO: 23 - DNA, 57 bp, Homo sapiens
atggagtttgggctgagctgggttttccttgttgctatttttaaaggtgtccagtgt

SEQ ID NO: 24 - DNA, 57 bp, Homo sapiens
atggagtttgggctgagctgggttttccttgttgttattttacaaggtgtccagtgt

SEQ ID NO: 25 - DNA, 57 bp, Homo sapiens
atgaaacacctgtggttcttcctcctgctggtggcagctcccagatgggtcctgtcc

SEQ ID NO: 26 - DNA, 57 bp, Homo sapiens
atgaaacacctgtggttcttcctcctcctggtggcagctcccagatgggtcctgtcc

SEQ ID NO: 27 - DNA, 57 bp, Homo sapiens
atgaagcacctgtggttcttcctcctgctggtggcggctcccagatgggtcctgtcc

SEQ ID NO: 28 - DNA, 57 bp, Homo sapiens
atgaaacatctgtggttcttccttctcctggtggcagctcccagatgggtcctgtcc

SEQ ID NO: 29 - DNA, 57 bp, Homo sapiens
atggggtcaaccgccatcctcgccctcctcctggctgttctccaaggagtctgtgcc

SEQ ID NO: 30 - DNA, 60 bp, Homo sapiens
atgtctgtctccttcctcatcttcctgcccgtgctgggcctcccatggggtgtcctgtca

SEQ ID NO: 31 - DNA, 66 bp, Homo sapiens
atggacatgagggtccccgctcagctcctggggctcctgctgctctggctcccaggtgccaaatgt

SEQ ID NO: 32 - DNA, 66 bp, Homo sapiens
atggacatgagggtccccgctcagctcctggggctcctgctgctctggctcccaggtgccagatgt

SEQ ID NO: 33 - DNA, 60 bp, Homo sapiens
atgagggtccccgctcagctcctggggctcctgctgctctggctcccaggtgccagatgt

SEQ ID NO: 34 - DNA, 66 bp, Homo sapiens
atggacatgagggtccccgctcagctcctggggctcctgctgctctggttcccaggttccagatgc

SEQ ID NO: 35 - DNA, 66 bp, Homo sapiens
atggacatgagagtcctcgctcagctcctggggctcctgctgctctgtttcccaggtgccagatgt

SEQ ID NO: 36 - DNA, 66 bp, Homo sapiens
atggacatgagggtccccgctcagctcctggggctcctgctgctctggttcccaggtgccaggtgt

SEQ ID NO: 37 - DNA, 66 bp, Homo sapiens
atggacatgagggtccctgctcagctcctgggactcctgctgctctggctcccagataccagatgt

SEQ ID NO: 38 - DNA, 66 bp, Homo sapiens
atggacatgagggtccctgctcagctcctggggctcctgctgctctggctctcaggtgccagatgt

SEQ ID NO: 39 - DNA, 66 bp, Homo sapiens
atggacatgagggtccccgctcagctcctggggctcctgctactctggctccgaggtgccagatgt

SEQ ID NO: 40 - DNA, 66 bp, Homo sapiens
atggacatgagggtccccgctcagctcctggggcttctgctgctctggctcccaggtgccagatgt

SEQ ID NO: 41 - DNA, 66 bp, Homo sapiens
atggacatgagggtccccgctcagctcctggggctcctactgctctgggtcccaggtgccagatgt

SEQ ID NO: 42 - DNA, 66 bp, Homo sapiens
atggacatgagggtgcccgctcagcgcctggggctcctgctgctctggttcccaggtgccagatgt

SEQ ID NO: 43 - DNA, 60 bp, Homo sapiens
atgaggctccttgctcagcttctggggctgctaatgctctgggtccctggatccagtggg

SEQ ID NO: 44 - DNA, 60 bp, Homo sapiens
atgaggctccctgctcagctcctggggctgctaatgctctgggtctctggatccagtggg

SEQ ID NO: 45 - DNA, 60 bp, Homo sapiens
atgaggctccctgctcagctcctggggctgctaatgctctggatccctggatccagtgcg

SEQ ID NO: 46 - DNA, 60 bp, Homo sapiens
atgaggctccctgctcagctcctggggctgctaatgctctgggtcccaggatccagtggg

SEQ ID NO: 47 - DNA, 60 bp, Homo sapiens
atggaagccccagctcagcttctcttcctcctgctactctggctcccagataccaccgga

SEQ ID NO: 48 - DNA, 60 bp, Homo sapiens
atggaagccccagcgcagcttctcttcctcctgctactctggctcccagataccactgga

SEQ ID NO: 49 - DNA, 60 bp, Homo sapiens
atggaaaccccagcgcagcttctcttcctcctgctactctggctcccagataccaccgga

SEQ ID NO: 50 - DNA, 69 bp, Homo sapiens
atggaaccatggaagccccagcacagcttcttcttcctcctgctactctggctcccagataccaccgga

SEQ ID NO: 51 - DNA, 60 bp, Homo sapiens
atggtgttgcagacccaggtcttcatttctctgttgctctggatctctggtgcctacggg

SEQ ID NO: 52 - DNA, 60 bp, Homo sapiens
atggggtcccaggttcacctcctcagcttcctcctcctttggatctctgataccagggca

SEQ ID NO: 53 - DNA, 57 bp, Homo sapiens
atgttgccatcacaactcattgggtttctgctgctctgggttccagcctccaggggt

SEQ ID NO: 54 - DNA, 51 bp, Homo sapiens
atgagggcttggatcttctttctgctctgcctggccgggcgcgccctcgca

SEQ ID NO: 55 - DNA, 57 bp, Homo sapiens
atggagtttgggctgagctgggttttcctcgttgctctttttagaggtgtccagtgt

SEQ ID NO: 56 - DNA, 75 bp, Homo sapiens
atgggggtactgctcacacagaggacgctgctcagtctggtccttgcactcctgtttccaagcatggcgagcatg

SEQ ID NO: 57 - DNA, 48 bp, Vesicular stomatitis virus
atgaagtgccttttgtacttagcctttttattcattggggtgaattgc

SEQ ID NO: 58 - DNA, 63 bp, Mus musculus
atggagacagacacactcctgctatgggtgctgctgctctgggttccaggttccactggtgac

SEQ ID NO: 59 - DNA, 57 bp, Homo sapiens
atgggctggtcctgcatcatcctgtttctggtggccacagccaccggtgtccattct

SEQ ID NO: 60 - DNA, 48 bp, Bombyx mori
atgaagcctatatttttggtattactcgtcgttacaagcgcctacgct

SEQ ID NO: 61 - DNA, 63 bp, Artificial Sequence
  Ariticfically created leader peptide sequence; Barash et al. (2002) Biochem. Biophys. Res. Commun. 294 835
atgtggtggcgcctgtggtggctgctgctgctgctgctgctgctgtggcccatggtgtgggcc

SEQ ID NO: 62 - DNA, 51 bp, Homo sapiens
atgccgctgctgctactgctgcccctgctgtgggcaggggccctggctatg

SEQ ID NO: 63 - DNA, 69 bp, Homo sapiens
atggatgcaatgaagagagggctctgctgtgtgctgctgctgtgtggagcagtcttcgtttcgcccagc

SEQ ID NO: 64 - DNA, 54 bp, Homo sapiens
atggctttcctctggctcctctcctgctgggccctcctgggtaccaccttcggc

SEQ ID NO: 65 - DNA, 45 bp, Homo sapiens
atgaatctacttctgatccttacctttgttgcagctgctgttgct

SEQ ID NO: 66 - DNA, 60 bp, Homo sapiens
atgtacaggatgcaactcctgtcttgcattgcactaagtcttgcacttgtcacaaacagt

SEQ ID NO: 67 - DNA, 51 bp, Gaussia princeps
atgggagtgaaagttctttttgcccttatttgtattgctgtggccgaggcc

SEQ ID NO: 68 - DNA, 54 bp, Homo sapiens
atgaagtgggtaacctttatttcccttctttttctctttagctcggcttattcc

SEQ ID NO: 69 - DNA, 48 bp, Influenza A virus
atgaagaccatcattgctttgagctacattttctgtctggttctcggc

SEQ ID NO: 70 - DNA, 72 bp, Homo sapiens
atggccctgtggatgcgcctcctgcccctgctggcgctgctggccctctggggacctgacccagccgcagcc

SEQ ID NO: 71 - protein, 19 aa, Homo sapiens
MDWTWRILFLVAAATGAHS

SEQ ID NO: 72 - protein, 19 aa, Homo sapiens
MDWTWRILFLVAAATGAHS

SEQ ID NO: 73 - protein, 19 aa, Homo sapiens
MDWTWSILFLVAAATGAHS

SEQ ID NO: 74 - protein, 19 aa, Homo sapiens
MDCTWRILFLVAAATGTHA

SEQ ID NO: 75 - protein, 19 aa, Homo sapiens
MDWTWRILFLVAAVTDAYS

SEQ ID NO: 76 - protein, 19 aa, Homo sapiens
MDWTWRVFCLLAVAPGAHS

SEQ ID NO: 77 - protein, 19 aa, Homo sapiens
MDWIWRILFLVGAATGAHS

SEQ ID NO: 78 - protein, 19 aa, Homo sapiens
MDWTWRFLFVVAAATGVQS

SEQ ID NO: 79 - protein, 19 aa, Homo sapiens
MDTLCYTLLLLTTPSWVLS

SEQ ID NO: 80 - protein, 19 aa, Homo sapiens
MDILCSTLLLLTVPSWVLS

SEQ ID NO: 81 - protein, 19 aa, Homo sapiens
MELGLSWVFLVAILEGVQC

SEQ ID NO: 82 - protein, 19 aa, Homo sapiens
MEFGLSWVFLVAIIKGVQC

SEQ ID NO: 83 - protein, 19 aa, Homo sapiens
MEFGLSWIFLAAILKGVQC

SEQ ID NO: 84 - protein, 19 aa, Homo sapiens
MEFGLSWVFLVAILKGVQC

SEQ ID NO: 85 - protein, 19 aa, Homo sapiens
MELGLRWVFLVAILEGVQC

SEQ ID NO: 86 - protein, 19 aa, Homo sapiens
MEFGLSWLFLVAILKGVQC

SEQ ID NO: 87 - protein, 19 aa, Homo sapiens
MEFGLSWLFLVAILKGVQS

SEQ ID NO: 88 - protein, 19 aa, Homo sapiens
MEFGLSWVFLVALLRGVQC

SEQ ID NO: 89 - protein, 19 aa, Homo sapiens
MELGLCWVFLVAILEGVQC

SEQ ID NO: 90 - protein, 19 aa, Homo sapiens
MEFWLSWVFLVAISKGVQC

SEQ ID NO: 91 - protein, 19 aa, Homo sapiens
MEFGLSWVFLVAIFKGVQC

SEQ ID NO: 92 - protein, 19 aa, Homo sapiens
MEFGLSWVFLVVILQGVQC

SEQ ID NO: 93 - protein, 19 aa, Homo sapiens
MKHLWFFLLLVAAPRWVLS

SEQ ID NO: 94 - protein, 19 aa, Homo sapiens
MGSTAILALLLAVLQGVCA

SEQ ID NO: 95 - protein, 20 aa, Homo sapiens
MSVSFLIFLPVLGLPWGVLS

SEQ ID NO: 96 - protein, 22 aa, Homo sapiens
MDMRVPAQLLGLLLLWLPGAKC

SEQ ID NO: 97 - protein, 22 aa, Homo sapiens
MDMRVPAQLLGLLLLWLPGARC

SEQ ID NO: 98 - protein, 20 aa, Homo sapiens
MRVPAQLLGLLLLWLPGARC

SEQ ID NO: 99 - protein, 22 aa, Homo sapiens
MDMRVPAQLLGLLLLWFPGSRC

SEQ ID NO: 100 - protein, 22 aa, Homo sapiens
MDMRVLAQLLGLLLLCFPGARC

SEQ ID NO: 101 - protein, 22 aa, Homo sapiens
MDMRVPAQLLGLLLLWFPGARC

SEQ ID NO: 102 - protein, 22 aa, Homo sapiens
MDMRVPAQLLGLLLLWLPDTRC

SEQ ID NO: 103 - protein, 22 aa, Homo sapiens
MDMRVPAQLLGLLLLWLSGARC

SEQ ID NO: 104 - protein, 22 aa, Homo sapiens
MDMRVPAQLLGLLLLWLRGARC

SEQ ID NO: 105 - protein, 22 aa, Homo sapiens
MDMRVPAQLLGLLLLWVPGARC

SEQ ID NO: 106 - protein, 22 aa, Homo sapiens
MDMRVPAQRLGLLLLWFPGARC

SEQ ID NO: 107 - protein, 20 aa, Homo sapiens
MRLLAQLLGLLMLWVPGSSG

SEQ ID NO: 108 - protein, 20 aa, Homo sapiens
MRLPAQLLGLLMLWVSGSSG

SEQ ID NO: 109 - protein, 20 aa, Homo sapiens
MRLPAQLLGLLMLWIPGSSA

SEQ ID NO: 110 - protein, 20 aa, Homo sapiens
MRLPAQLLGLLMLWVPGSSG

SEQ ID NO: 111 - protein, 20 aa, Homo sapiens
MEAPAQLLFLLLLWLPDTTG

SEQ ID NO: 112 - protein, 20 aa, Homo sapiens
METPAQLLFLLLLWLPDTTG

SEQ ID NO: 113 - protein, 23 aa, Homo sapiens
MEPWKPQHSFFFLLLLWLPDTTG

SEQ ID NO: 114 - protein, 20 aa, Homo sapiens
MVLQTQVFISLLLWISGAYG

SEQ ID NO: 115 - protein, 20 aa, Homo sapiens
MGSQVHLLSFLLLWISDTRA

SEQ ID NO: 116 - protein, 19 aa, Homo sapiens
MLPSQLIGFLLLWVPASRG

SEQ ID NO: 117 - protein, 17 aa, Homo sapiens
MRAWIFFLLCLAGRALA

SEQ ID NO: 118 - protein, 19 aa, Homo sapiens
MEFGLSWVFLVALFRGVQC

SEQ ID NO: 119 - protein, 25 aa, Homo sapiens
MGVLLTQRTLLSLVLALLFPSMASM

SEQ ID NO: 120 - protein, 16 aa, Vesicular stomatitis virus
MKCLLYLAFLFIGVNC

SEQ ID NO: 121 - protein, 21 aa, Mus musculus
METDTLLLWVLLLWVPGSTGD

SEQ ID NO: 122 - protein, 19 aa, Homo sapiens
MGWSCIILFLVATATGVHS

SEQ ID NO: 123 - protein, 16 aa, Bombyx mori
MKPIFLVLLVVTSAYA

SEQ ID NO: 124 - protein, 21 aa, Artificial Sequence
  Ariticfically created leader peptide sequence; Barash et al. (2002) Biochem. Biophys. Res. Commun. 294 835
MWWRLWWLLLLLLLLWPMVWA

SEQ ID NO: 125 - protein, 17 aa, Homo sapiens
MPLLLLLPLLWAGALAM

SEQ ID NO: 126 - protein, 23 aa, Homo sapiens
MDAMKRGLCCVLLLCGAVFVSPS

SEQ ID NO: 127 - protein, 18 aa, Homo sapiens
MAFLWLLSCWALLGTTFG

SEQ ID NO: 128 - protein, 15 aa, Homo sapiens
MNLLLILTFVAAAVA

SEQ ID NO: 129 - protein, 20 aa, Homo sapiens
MYRMQLLSCIALSLALVTNS

SEQ ID NO: 130 - protein, 17 aa, Gaussia princeps
MGVKVLFALICIAVAEA

SEQ ID NO: 131 - protein, 18 aa, Homo sapiens
MKWVTFISLLFLFSSAYS

SEQ ID NO: 132 - protein, 16 aa, Influenza A virus
MKTIIALSYIFCLVLG

SEQ ID NO: 133 - protein, 24 aa, Homo sapiens
MALWMRLLPLLALLALWGPDPAAA

SEQ ID NO: 134 - DNA, 78 bp, Homo sapiens
atggatctcatgtgcaagaaaatgaagcacctgtggttcttcctcctgctggtggcggctcccagatgggtcctgtcc

SEQ ID NO: 135 - protein, 26 aa, Homo sapiens
MDLMCKKMKHLWFFLLLVAAPRWVLS

SEQ ID NO: 136 - DNA, 160 bp, Homo sapiens
atggagtttgggctgagctggctttttcttgtggctattttaaaaggtaattcatggagaaatagaaaaattgagtgtgaatggataagagtgagagaaacagtggatacgtgtggcagtttctgaccagggtttctttttgtttgcaggtgtccagtgt

SEQ ID NO: 137 - DNA, 247 bp, Homo sapiens
atggaaaccccagcgcagcttctcttcctcctgctactctggctcccaggtgaggggaacatgggatggttttgcatgtcagtgaaaaccctctcaagtcctgttacctggcaactctgctcagtcaatacaataattaaagctcaatataaagcaataattctggctcttctgggaagacaatgggtttgatttagattacatgggtgacttttctgttttatttccaatctcagataccaccgga